(19) United States
(12) Patent Application Publication
(10) Pub. No.: US 2007/0173886 A1
(43) Pub. Date: Jul. 26, 2007

(54) Method and system for external counterpulsation

(75) Inventors: Benny Rousso, Rishon LeZion (IL); Yuval Avni, Givatayim (IL); Eliahu Eliachar, Haifa (IL); Nir Lilach, Kfar Yehoshua (IL)

Correspondence Address: PEARNE & GORDON LLP, 1801 EAST 9TH STREET, SUITE 1200, CLEVELAND, OH, 44114-3108, US

(73) Assignee: FLOWMEDIC LIMITED, Hamilton (BM)

(21) Appl. No.: 10/597,613

(22) PCT Filed:
(86) PCT No.: PCT/IL05/00150

(30) Foreign Application Priority Data
Feb. 4, 2004 (IL) 160214
Sep. 26, 2004 (IL) 164286

(51) Int. Cl. A61B 17/00

(52) U.S. Cl. 606/203

(57) ABSTRACT

A system and method for modulating cardiac blood flow of a patient by applying intermittent pressure on a limb of the patient, the system comprising a limb attachment, substantially surrounding the limb perimeter and an actuator pulling and releasing a flat member; a sensor for determining a physiological parameter relating to the patient; and a processing unit for receiving data from the sensor and issuing commands to the actuator.

### RELATED APPLICATIONS

The present invention claims priority from Israeli patent application Ser. No. 160214 titled A PORTABLE DEVICE FOR THE ENHANCEMENT OF CIRCULATION OF BLOOD AND LYMPH FLOW IN A LIMB, filed on 4 Feb. 2004, and from Israeli patent application Ser. No. 164286 titled A PORTABLE DEVICE FOR THE ENHANCEMENT OF CIRCULATION filed on 26 Sep. 2004. The full contents of the abovementioned applications are incorporated herein by reference.

The present invention is a continuation in part of international patent application Ser. No. PCT/IL2004/00487 titled A PORTABLE DEVICE FOR THE ENHANCEMENT OF CIRCULATION OF BLOOD AND LYMPH FLOW IN A LIMB filed on 9 Jun. 2004, the full content of which is incorporated herein is by reference, and which claims priority from Israeli patent application No. 160185 titled A PORTABLE DEVICE FOR THE ENHANCEMENT OF CIRCULATION OF BLOOD AND LYMPH FLOW IN A LIMB, filed on Feb. 2, 2004. PCT/IL2004/00487 is a continuation in part of international patent application Ser. No. PCT/IL02/00157 titled A PORTABLE DEVICE FOR THE ENHANCEMENT OF THE CIRCULATION AND FOR THE PREVENTION OF STASIS RELATED DEEP VEIN THROMBOSIS DVT filed on 3 Mar. 2002, which claims priority from Israeli patent application No. 141824 titled A PORTABLE DEVICE FOR THE ENHANCEMENT OF THE CIRCULATION AND FOR THE PREVENTION OF STASIS RELATED DEEP VEIN THROMBOSIS (DVT) filed on Mar. 5, 2001. The full contents of all the abovementioned applications are also incorporated herein by reference.

### BACKGROUND OF THE INVENTION

1. Field of the Invention

The present invention relates to medical devices and methods in general, and to a method and system for external counterpulsation in particular.

2. Discussion of the Related Art

Ischemia is generally a condition in which the blood flow, and thus oxygen, is restricted to a part of the body. Cardiac ischemia is the name for lack of blood flow and oxygen to the heart muscle. When the heart contracts during the systole, the coronary vasculature is squeezed by the powerful contraction of the heart muscle, limiting the flow of blood to and within the myocardium. The myocardium must therefore receive most of its oxygenated blood during the diastole, when the coronary arteries are most receptive to flow. Ischemic heart disease is the term given to heart problems caused by narrowed heart arteries. When arteries are narrowed, less blood and oxygen reaches the heart muscle. Ischemic heart disease is also called coronary artery disease and coronary heart disease. These diseases can ultimately lead to heart attack. Ischemia often causes chest pain or discomfort known as angina pectoris.

Peripheral vascular disorders include venous, arterial or combined arteriovenous disorders. Venous thrombosis may seriously affect superficial or deep veins. Over time, serious conditions may develop to include edema, pain, stasis pigmentation, dermatitis, ulceration, and the like. Serious cases of venous thrombosis may lead to phlegmasia cerulea dolens in which the extremities of the patient turns blue and may lead to gangrene and death. Various other ailments and conditions are likely to result from complications of venous thrombosis. Arterial vascular disorders such as peripheral arterial occlusion may result in acute ischemia manifested in cold, painful, and discolored extremities. In acute cases, the locations distal to the obstruction will be absent of pulse. Chronic occlusion will be manifested in the patient being able to walk to a lesser distance as the diseases progresses, causing unrelenting pain to the extremities, compromising tissue viability and leading to gangrene.

Enhanced external counterpulsation (EECP) is a non-invasive, atraumatic procedure that can reduce the symptoms of angina pectoris, and other ischemia-related diseases, as well as for other peripheral vascular diseases, presumably by increasing coronary bloodflow in ischemic areas of the heart and generally improving blood and lymph flow in the body of the patient.

Currently available EECP devices comprise one or more pairs of air-pressurized cuffs attached to the person's legs at the calves, thighs, or hips. During EECP treatment rapid and precisely timed squeezing of the muscles sends a wave of blood and pressure that travels toward the heart through both veins and arteries. The compression ensures that the waves generated in the most distant calf muscles can pass under the next cuffs lower thighs and be reinforced rather than be trapped by the second and third compressions of the sequence. The result of the combined compressions is an increase in venous return, and an augmentation, or enhancement of diastolic pressure, which improves myocardial perfusion. The beneficial effects of EECP on perfusion of the ischemic myocardium in patients with coronary artery disease appear to be sustained between treatments, and may persist long after completion of a course of therapy.

Since existing EECP devices utilize pressurized air cuffs, large and costly pumping equipment is required, which makes such presently available device extremely cumbersome, expensive, and noisy. A dedicated space is required for the now known EECP device and therefore the treatment can only be suggested in health care institutes, and not in the patient's home.

Accordingly it is the object of the present invention to provide an EECP device for the enhancement of cardiac and peripheral blood flow which is portable, self-contained and easily carried, small and lightweight, is easy to manufacture and has a low cost. Such device will have enhanced energetic abilities enabling the efficient suction of blood and lymph through the arterial vessels, and enhancing blood and lymph flow, in general. A further object of the invention is to provide such a device with sensors for gathering data of the heart beat cycle or physiological condition of the patient, and a controller for activating the compressing devices in accordance with the gathered data.

It is further an object of the present invention to provide such a device which is simple to operate by a lay person without any special training in the field of medicine, is easily strapped over or attached to a limb and can be easily adjusted to fit persons of any size.

Other advantages of the invention will be apparent from the description that follows.

### SUMMARY OF THE PRESENT INVENTION

The disclosed invention presents a novel system and method for modulating cardiac blood flow of a patient. The system of the proposed invention intermittently applies and releases pressure on a limb of the patient, the system comprises one or more limb attachment comprises an at least one generally encircling member substantially surrounding the limb perimeter in at least one location, at least one actuator for pulling and releasing the flat member. The system further comprises one or more processing unit for issuing commands to the at least one actuator. The commands are determined based on one or more physiological parameters of the patient. The system further comprising one or more connections to external sensors measuring the physiological parameters of the patient. The system further comprising one or more sensors for measuring physiological parameters of the patient. The actuator is a mechanical actuator, or the actuator is attached to the patient. The system where each flat members is a strap, a flap, a closure, or a sleeve. The system wherein the physiological parameter is sensed by an electrocardiograph or photoplathismography device, and the physiological parameter is one ore more of the following: heart rate; blood pressure; blood vessels; movement; temperature; sweat amount; sweat composition. The system applies pressure such as to modulate cardiac blood flow by increasing the arterial and coronary pressure during the diastole, or by increasing the aorta and peripheral arteries pressure during the systole. The system improves peripheral circulation such as to reduce peripheral resistance. The system wherein the pressure is applied to improve venous return. The system applies pressure gradients such as to promote vasodilatation, or the system applies falling pressure gradient such as to produce venous suction effect, or the system applies pressure with high gradients such as to create sheer forces along the arteries, promoting coronary dilatation. The system applies pressure in several locations along the limbs wherein the pressure is applied substantially in a synchronized fashion. The intermittent pressure applications or releases are synchronized. The delay between intermittent pressure applications is less than 500 milliseconds, or less than 100 milliseconds. The intermittent pressure is applied sequentially and optionally the sequence starts from distal regions. The system being responsive to one ore more physiological event. Optionally, the physiological event is used as a trigger for applying and releasing intermittent pressure on the limb of the patient. Optionally, the processing unit analyzes the physiological parameter and controls the system activity accordingly. The commands issued by the processing unit relate to any of the following: pressure levels, pressure durations, no-pressure duration, pressure rise time, pressure fall time, pressure delays among locations, rest periods, sequence of pressure levels, or pressure delay from physiological event. The pressure levels applied on limb subject to the processing unit control can be at any level that can be reached by the system. The limb attachment further comprises a sensor. The sensor being an external sensor. The sensor senses pressure or force or temperature or impedance or can be a plethysmography sensor. The pressure or the force or the temperature, or the impedance sensed, or the plethysmography sensor, are used in determining the commands issued by the processing unit. The commands are issued according to a timing mechanism, or according to the heart beat of the patient every predetermined number of heart beat cycles. The limb attachments are attached to a foot, a calf, a shin, a hip, an upper arm, or a forearm. The system optionally comprises one or more pairs of limb attachments, each pair of limb attachments is symmetrically attached to the body of the patient. The system is used for treating heart failure, or coronary disease or angina pectoris. The system optionally applies pressure on the limb by applying a pre-determined force, or by applying a pre-determined pressure. The volume required for the actuator used to produce the pressure at each limb attachment is less than 2000 cc, or less than 1000 cc. The weight required for the actuator used to produce the pressure at each limb attachment is less than 3 Kg, or less than 1.5 Kg or less than 1 Kg. The pressure rise time is less than 1 second, or less than 300 milliseconds, or less than 100 milliseconds or less than 50 milliseconds. The pressure fall time is less than 1 second, or less than 300 milliseconds, or less than 100 milliseconds or less than 50 milliseconds. The pressure is within the range of 15-400 mmHg, or within the range of 25-150 mmHg, or within the range of 40-100 mmHg. The pressure application duration is less than 3 seconds, or less than 1 second, or less than 300 milliseconds, or less than 100 milliseconds. Alternatively, the pressure application duration is longer than one heart beat, 5 heart beats, 30 heart beats, and 100 heart beats. The actuator comprises a motor and one or more mechanical elements such as rods, tooth wheels, eccentric wheels, etc. The system further comprising one or more data logging unit. The data logging unit stores activity information of the system comprising time, pressure, usage information. In the case when the commands are determined based on one or more physiological parameters, the physiological parameters are stored by the data logging unit as well. The system can further comprise an analysis unit for data reviewing and analysis.

Another aspect of the disclosed invention includes a method for modulating cardiac blood flow of a patient by applying intermittent pressure on a limb of the patient, the method comprising the steps of receiving one or more physiological parameters from one or more sensors associated with the patient; determining the beginning of diastole based on the physiological parameters; issuing one or more commands to one or more actuators associated with an at least one limb attachment, said actuators causing the beginning or end of application of pressure by the limb attachment on the limb, thereby changing the circumference of the limb of the patient thus modulating the cardiac blood flow of the patient; and

executing the command by the actuators. The pressure is applied to the body of the patient starting at distal limb attachments. The pressure is applied only during systole. The pressure is applied symmetrically to the body of the patient. The pressure is applied by shortening of one or more straps circulating the limb, or by pressing with one or more movable plates or by pressing with one or more flaps moving in and out of a housing. The method further comprising a step of storing data, the data includes activity information comprising time, pressure and usage information, or the data includes physiological data. The method further comprising the step of measuring the at least one physiological parameter. The application of pressure by the at least one limb attachment on the limb can be performed intermittently. The method further comprises a step of evaluating the physical condition of the patient prior to issuing the at least one command. The pressure is applied only during systole. The pressure is applied symmetrically to the body of the patient. The pressure is applied to the body of the patient starting at distal limb attachments.

### BRIEF DESCRIPTION OF THE DRAWINGS

The present invention will be understood and appreciated more fully from the following detailed description taken in conjunction with the drawings in which:

FIG. 1 is a pictorial illustration of the device of the present invention strapped to the calf of a sitting person, in accordance with the present invention;

FIG. 2A is a side external view of a preferred anterior box embodiment of the present device, in which squeezing the limb muscles is performed by intermittent shortening the circumference of a loop created by an assembly body and strap, in accordance with the present invention;

FIG. 2B is a side view illustration of an posterior box embodiment in which the assembly box is the active intermittent compressing part placed against the calf muscles, in accordance with the present invention;

FIG. 3A is a cross section of a device in accordance with the embodiment of FIG. 2A, showing a first internal mechanism of the assembly box, in accordance with the present invention;

FIG. 3B is a top view of the device of FIG. 3A, in accordance with the present invention;

FIG. 3C depicts a modified mechanism of the embodiment of FIGS. 3A and 3B, in accordance with the present invention;

FIG. 4A is pictorial representation of an alternative mechanism for the embodiment of FIG. 2A using electromagnetic motor, a centrally hinged rotating rectangular plate and a longitudinal bar connecting both sides of the strap, in accordance with the present invention;

FIG. 4B and 4C are side and top view respectively of the embodiment presented in FIG. 4A, in accordance with the present invention;

FIG. 5A and 5B depict yet another mechanism for the embodiment of FIG. 2A using an enhanced power transmission by means of an “L” shaped lever bar, in accordance with the present invention;

FIG. 6 is a side view of yet another embodiment of a device in accordance with the present invention, in accordance with the present invention;

FIG. 7 is a top view of a device in accordance with the anterior box embodiment of FIG. 2B showing the internal mechanism of the assembly box, in accordance with the present invention;

FIGS. 8 depicts an enhanced embodiment of the present invention, referred to as reverse propulsion embodiment, in accordance with the present invention:

FIG. 8A and 8B are rear and frontal perspective views, respectively, of a device in accordance with the reverse propulsion embodiment, in accordance with the present invention;

FIG. 8C is a rear perspective view of the reverse propulsion embodiment of FIGS. 8A and 8B in an upside down position with back cover removed to show internal components in loose strap state, in accordance with the present invention;

FIG. 8D is a rear perspective view of reverse propulsion embodiment as in FIG. 8C with both frontal and back covers removed, showing internal components in contracted state, in accordance with the present invention;

FIGS. 8E and 8F and are a rear and frontal perspective views, respectively, of the reverse propulsion embodiment in horizontal position with both covers removed, in accordance with the present invention;

FIG. 8G is a perspective view of the main mechanism, referred to as a reverse propulsion mechanism, responsible for actuating transitions between relaxed and contracted states of the strap, in accordance with the present invention;

FIG. 8H is a perspective view of the force adjustment mechanism of the reverse propulsion embodiment, in accordance with the present invention;

FIGS. 9 describe yet another enhanced embodiment of the present invention, in accordance with the present invention:

FIG. 9A is a top elevational perspective external view of the embodiment, in accordance with the present invention;

FIG. 9B is an elevational perspective view of the embodiment of FIG. 9A with top cover and side walls removed to show internal components, in accordance with the present invention;

FIG. 9C is an eleveational perspective view of the embodiment of FIG. 9A with top cover, side walls and rollers removed, in accordance with the present invention;

FIG. 9D is a sequence of side views of the ratchet mechanism of the embodiment illustrated in FIG. 9B, as function of time, demonstrating the operation of the ratchet mechanism, in accordance with the present invention;

FIG. 9E is a time sequence of cross sectional views of the clutch of the embodiment of FIG. 9B at a plane perpendicular to the rotation axis, demonstrating the operation of the clutch, in accordance with the present invention;

FIG. 9F is an illustration of a typical user interface of the embodiment illustrated in FIGS. 9A-9C, in accordance with the present invention;

FIGS. 10A and 10B are typical pressure profiles obtained by a device of the present invention and a commercially available IPC device, respectively, in accordance with the present invention;

FIG. 11 is a schematic illustration of a patient using the system in accordance with the present invention;

FIG. 12 is a block diagram representing the steps of using the system accordance with the present invention; and

FIGS. 13A, 13B and 13C are examples of energetic patterns of the apparatus and method of the present invention, in accordance with the present invention.

### DETAILED DESCRIPTION OF THE PREFERRED EMBODIMENT

A system for the intermittent compression of the extremities muscles for the enhancement of cardiac blood flow and lymph flow in a limb and the prevention of peripheral vascular diseases is disclosed. The system of the present invention is an external counter pulsation system, comprising a plurality of compressing elements, one or more sensors or other devices providing data related to the physiological condition of the patient, and a control system to coordinate the compression parameters of the compressing elements in accordance with the physiological conditions. More specifically, the sensor can be an electrocardiograph (ECG) device, detecting the diastole and systole of the patient and causing the compressing elements to compress at the diastole and loosen at the systole.

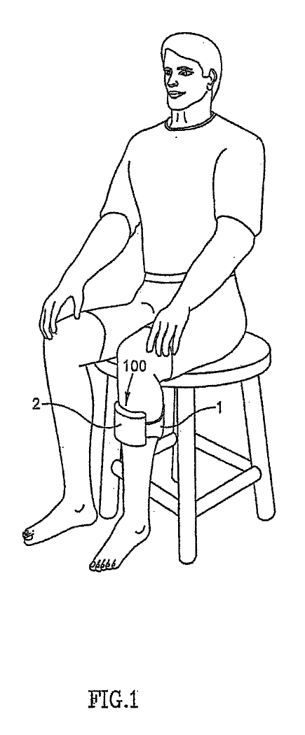

Referring now to FIGS. 1, 2, 3, 4, 5, 6, 7, 8, 9, and 10, presenting devices for the intermittent compression of the extremities muscles for the enhancement of blood and lymph flow in a limb. The present invention can be helpful in the prevention of Deep Vein Thrombosis (DVT), reduce lymph edema, prevent and reduce incidence and complications of diabetic as well as other arterial insufficiency states by applying periodic squeezing forces on a limb, in particular a lower limb. More specifically, the present invention relates to a portable, self contained, mechanical device for enhancing the blood in a limb, enhancing the lymph and venous return from a limb, specifically a lower limb, towards the heart, aiming at reducing the risk of DVT formation, edema formation, lymphedema, and improving the general circulation in a limb during periods of immobility, increased stasis as well as conditions of reduced circulation such as in diabetic patients, post surgical patients and the like. The present invention discloses a mechanical apparatus and the method of operation of the same having favorable energetic features allowing the operation of the apparatus at a maximum output with minimal energy input. The device and the method of operation of the present invention operates at a best energetic efficiency by utilizing low input energy having an energy saving machinery thus enhancing energy output, more specifically by utilizing energy source optimization, internal machinery energy saving features as well as tissue characteristics enhances the favorable energetic profile of the present apparatus as well as reducing the energy requirement of the apparatus. The present invention can also operate at different energetic profiles suitable for the multitude of purposes more specifically for enhancing venous, arterial as well as lymph flow through a limb.

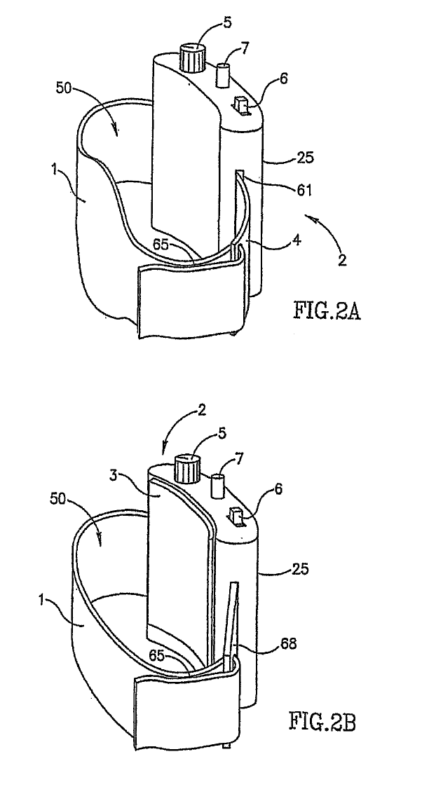

The portable device of the present invention, generally designated 100, is shown in FIG. 1, worn on the calf of a sitting person, Device 100 can be worn directly on the bare limb, or on a garment, such as trousers, worn by the person using the device. Device 100 comprises two main components, an assembly box 2 which contains all the machinery parts responsible for the device operation, and a strap 1 connected to said assembly box such as to form a closed loop (designated 50, see FIGS. 2) for encircling a person limb. The power supply for the device may be of the internal power supply type such as a rechargeable or non rechargeable low voltage DC batteries or an external power supply type such as an external power outlet connected via an AC/DC transformer such as a 3-12V 1Amp transformer, fed through electrical wires to a receptacle socket in the device (not shown). As shown in FIG. 1, strap 1 is preferably wide in the middle and narrow at the ends where it connects to assembly box 2. Strap 1 however may assume any other shape and form such as a constant width belt. The strap can be fabricated from any flexible material that is non-irritating to the skin, such as thin plastic, woven fabric and the like. Strap 1 can be fabricated from one material or alternatively can combine more than one material. For example, strap 1 can be made of both non stretchable material and stretchable material wherein such an arrangement may be dispose of a stretchable material for example rubber fabric in the center of the strap 1 and a non stretchable material such as plastic flanking the stretchable material and comprising the rest of the strap. Such an arrangement facilitates a more uniform stretch forces on the strap as well as preventing the slippage of the strap from the limb. According to the preferred embodiment shown in FIG. 1, hereinafter called the anterior box embodiment, strap 1 is placed against the muscles while assembly box 2 is placed against the calf bone. However, according to another embodiment of the present invention, hereinafter called the posterior box embodiment, assembly box 2 can be placed against the muscles.

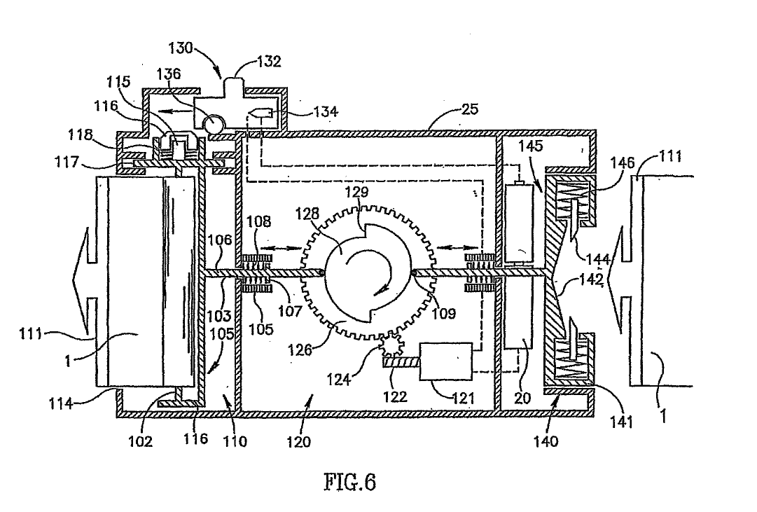

FIGS. 2A, 2B illustrate two possible embodiments of the device of the present invention. FIG. 2A represents a preferred embodiment of the present device, in which squeezing the limb muscles for promoting the increase of blood and lymph flow in the limb, is performed by pulling and releasing strap 1, thus, intermittently shortening the effective length of loop 50 encircling the limb. This embodiment is preferably used as an anterior box embodiment of the present invention. However, it will be easily appreciated that the device of FIG. 2A can be used as a posterior box embodiment as well. FIG. 2B presents another embodiment of the present device in which assembly box 2 is the active intermittent compressing part by means of mobile plate 3 attached to the box. This embodiment, which can be used only as a posterior box embodiment, will be explained in conjunction with FIG. 6.

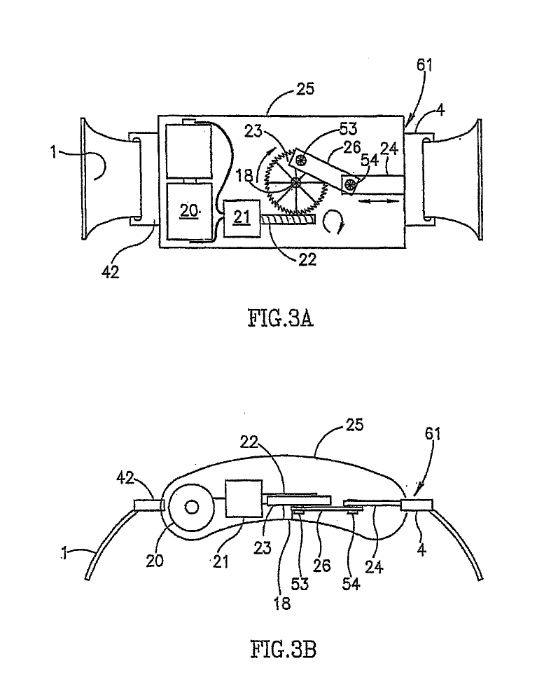

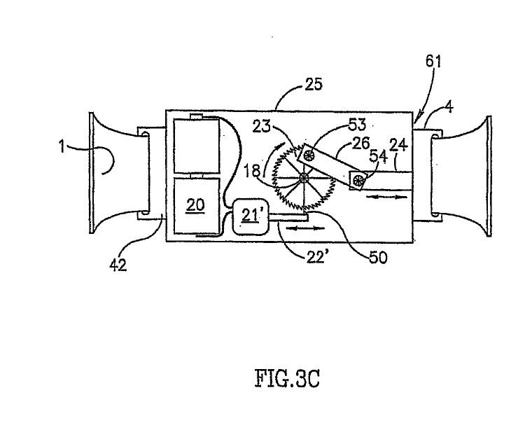

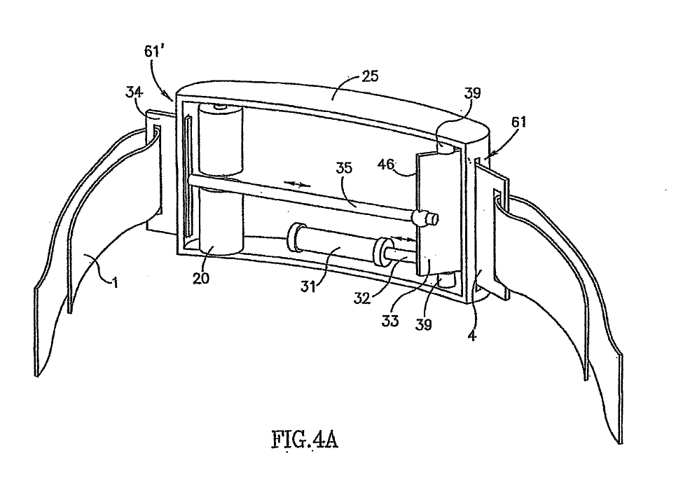

Turning back to FIG. 2A, assembly box 2 comprises a thin, curved flask-shaped casing 25 which contains all the parts of internal machinery responsible for intermittent pulling and releasing strap 1. Casing 25 is preferably fabricated from, but not limited to, a plastic molding, a light metal, or any other material which is light, non irritating to the skin, and cheep to produce. Strap 1 is connected at both its ends to assembly box 2 by means of two buckles 4 and 42 at the sides of casing 25 (buckle 42 not shown). At least one of said buckles (here buckle 4) is a mobile buckle, which can move in and out of dasing 25 through slit (opening) 61, thus pulling and relaxing strap 1 between a retracted and a relaxed positions. The retraction protraction motion shortens and lengthens the effective length of strap 1, thus causing intermittent compression of the underlying muscle and increasing the blood and lymph flow in the underlying vessels. Possible inner machinery responsible for activating the intermittent pulling of strap 1 is described in the following in conjunction with FIGS. 3 to 6. Strap 1 can be adjusted to fit the size of the limb, on which device 100 is to be operated, by having at least one of its ends free to move through its corresponding buckle, such that the strap can be pulled by said end for tightening the strap around said limb. Said end is then anchored in the appropriate position. In the example shown here, the strap is folded back on itself and the overlapping areas are fastened to each other by fastening means 65, such as Velcro™ strips, snap fasteners or any other fastening or securing means. Alternatively, said strap end can be secured to casing 25 by fastening means such as Velcro strips, opposite teeth-like protrusions both on casing 25 and on strap 1, and the like. The other end of strap 1 can be connected to its corresponding buckle either in a permanent manner by attaching means such as knots or bolts, or can be adjustable in a similar manner to what had been described above, allowing both ends to be pulled and anchored simultaneously for better fitting. Yet, in accordance with another embodiment of the invention, the strap can be wound around a retracting mechanism positioned at one side of casing 25. The free end of the strap can be provided with a buckle for allowing connection into the opposite side of casing 25 either by one of the aforementioned means described or by means of a quick connector. Outer casing box 25 also includes an on/off switch 6, a force regulator 5 for regulating the force exerted on the calf muscle by strap 1 and a rate regulator 7 for regulating the frequency of intermittent compressions. Alternatively, force regulator 5 and on/off switch 6 can be combined into one button. Force regulation can be obtained for example by way of controlling the length of the strap interval between retracted and protracted positions. The length interval between contracted and relaxed positions is preferably, but not limited to, 1-50 millimeters. Frequency regulation can be obtained by way of regulating, but not limited to, the speed of the inner machinery. A person skilled in the art will readily appreciate that the present invention can be used for the enhancement of both arterial and venous blood and lymph flow in a limb (upper and lower). The examples provided in the following discussion serve as an example and should not be construed as a limitation to the application of the preset invention.

Referring now to FIGS. 3A and 3B, there is shown a side view and a top view respectively of first inner machinery for the device of FIG. 2A. The numerical are corresponding in both drawings. According to this embodiment, one end of strap 1 is connected to assembly box 2 via a fixed fitting 42 by means such as bolts, knots glue, etc. The second end is connected via a movable buckle 4, which traverses slit 61 located at the side of casing 25. Buckle 4 can retract and protract through opening 61, as described above. Movable buckle 4 is connected to the inner machinery by means of attachment to a rigid push/pull rod 24. The inner machinery responsible for the motion of movable buckle 4 is herein described. Energy source 20 such as low voltage DC batteries, supplies electrical energy to an electrical motor 21 such as, but not limited to, a 3-12 V DC motor, via electrical contacts such as wires. Electric motor 21 converts electric energy into kinetic energy, spinning a spirally grooved (worm) central shaft 22. Shaft 22 is coupled to a (speed reduction) wheel 23, having complementary anti-spiral circumferential grooves or teeth, causing wheel 23 to revolve around its center which is fixed by axis 18 perpendicular to its surface. An elongated connector plate 26 is pivotally jointed at one end to off-center point 53 on wheel 23 and at its second end to rod 24 at point 54, such that the rotation of wheel 23 actuates plate 26 to intermittently push and pull rod 24, in a crankshaft manner. Consequently, mobile buckle 4 is intermittently pulled inward and outward casing 25 through slit 61, thus intermittently shortening the circumference of loop 50.

Modified machinery, represented in FIG. 3C, includes the following changes with reference to FIG. 3A and 3B. The electric motor 21 and spinning worm shaft 22 are replaced with an electromagnetic motor 21′ (such as a push-pull solenoid 191C distributed by Shindengen electric Ltd.) having a reciprocating central rod 22′ with an upwardly inclined spike-tooth projection 50 at its end. Rod 22′, via projection 50 is coupled to wheel 23, having complementary teeth. As reciprocating rod 22′ slightly protrudes from, and retracts into the motor body, projection 50 latches sequential teeth of wheel 23 as it protrudes and pulls wheel 23 as it retracts, causing wheel 23 to revolve around its axis. The mechanism of

FIG. 3C generates a large force output while minimizing the power input. Such machinery is very cost effective. The above description clearly shows how the internal mechanical machinery of the proposed device acts to intermittently shorten loop 50, culminating in intermittent compression of the leg or hand muscle and leading to increase of venous return and helping in the prevention of the formation of deep vein thrombosis.

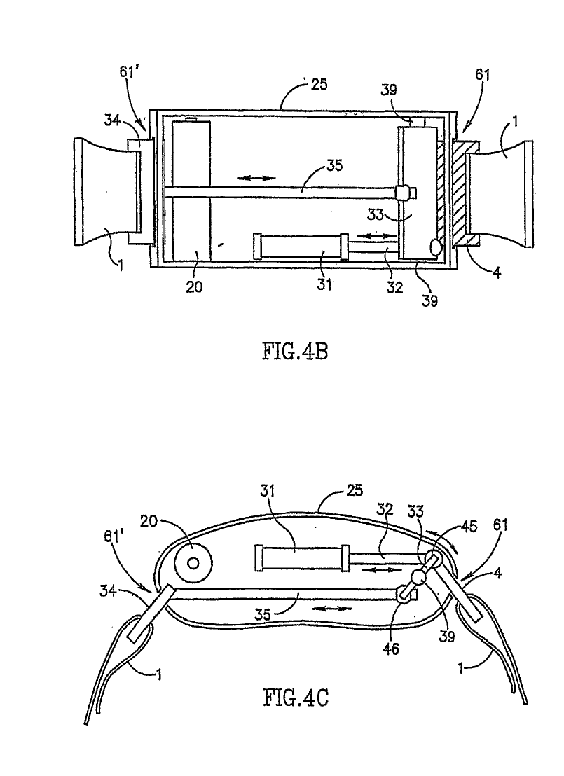

An alternative machinery embodiment for the device embodiment of FIG. 2A is shown in FIGS. 4A, 4B and 4C. FIG. 4A is a perspective drawing view showing the internal parts of assembly box 2 with the frontal part of casing 25 removed. FIG. 4B and 4C side and top view, respectively of the embodiment shown in FIG. 4A. According to this embodiment, both ends of strap 1 are connected to the inner machinery of assembly box 2 by means of two movable buckles 4 and 34, which can move inwardly and outwardly casing 25 through slits 61 and 61′, respectively. This alternative embodiment combines the following elements: A rectangular plate 33 positioned close to one side wall of casing 25, adjacent to slit 61. Plate 33 having two parallel rectangular surfaces, two narrow vertical edges, designated 45 and 46, and two narrow horizontal edges. Plate 33 is pivotally mounted at its narrow horizontal edges to the top and bottom walls of casing 25, by pivoting means 39, such as to allow rotational movement of the plate around the vertical axis connecting between pivoting means 39; A push-pull electromagnetic motor 31 (such as pull tubular solenoid 190 distributed by Shindengen electric Ltd.) connected via its reciprocating central rod 32 to one vertical edge (45) of the centrally hinged rectangular plate 33, at about mid point of said edge; A longitudinal rod 35 spans the length of casing 25. Said longitudinal rod 35 is connected at one end to the opposite vertical edge (46) of plate 33 and at its second end to movable buckle 34 positioned at the other side of casing 25. Centrally hinged rectangular plate 33 is thus connected on one side to the electromagnetic motor 31 via central rod 32, and on the other side to longitudinal rod 35 (as best seen in FIG. 4C). Movable buckle 4 is also connected to narrow edge 45 of plate 33 but extends outwardly, through slit 61, in the opposite direction to rods 32 and 35.

As can be best seen in FIG. 4C, the reciprocating movement of rod 32 causes plate 33 to turn back and forth around its central axis, preferably the angular displacement is in the range of 20 to 60 degrees. Consequently, buckles 4 (coupled directly to plate 33) and 34 (by means of connecting rod 35) are synchronously pulled and pushed inward and outward of casing 25, resulting in intermittent shortening of the limb encircling loop. This embodiment is advantageous because the longitudinal rod 35 allows both buckles 34 and 4 to approximate each other at the same time, thus enhancing the efficiency of the device (by enhancing the reciprocating displacement of electromagnetic motor 31) and requiring less energy.

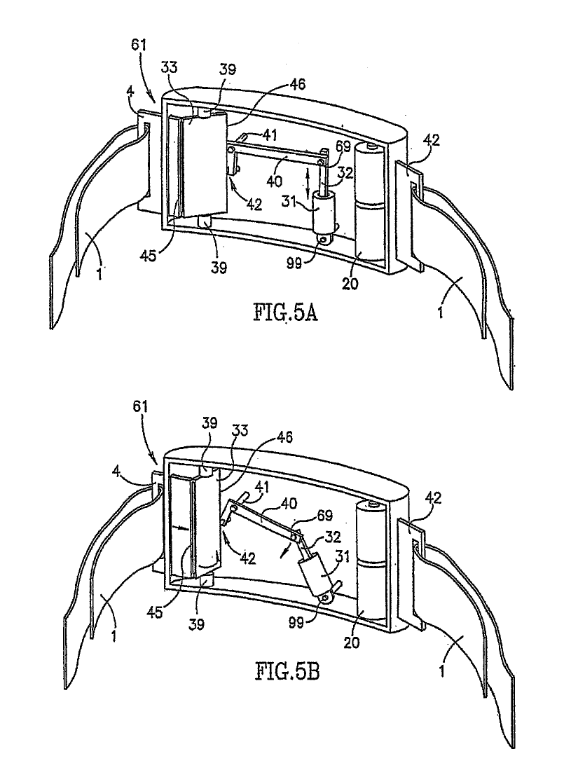

FIGS. 5A and 5B illustrate yet another alternative machinery for the device embodiment of FIG. 2A. The embodiment of FIGS. 5 also uses a pull-push electromagnetic motor as the driving force but allows force enhancement by the addition of an “L” shaped lever bar 40 to the said centrally displaced rod 32 of the embodiment shown in FIGS. 4. According to this embodiment, one edge of strap 1 is connected to fixed buckle 42 while the second end is connected to movable buckle 4 which transverse casing 25 through side slit 61. The movable buckle 4 is connected to centrally hinged rectangular plate 33 in a similar manner to what have been described in conjunction with FIGS. 4. In accordance with the present embodiment, electromagnetic motor 32 is pivotally mounted at its rear end to the base by pivoting means 99. The “L” shaped lever bar 40 pivotally mounted at its longer arm end to reciprocating rod 32 by pivoting means 39, and at its shorter arm end is attached to narrow edge 46 of plate 33, by attaching means 42, in a manner which allows it to slide up and down said edge. Such attaching means can be obtained, for example, by railing means such as a groove engraved along the edge of the short arm of lever 40 and a matching protruding railing extending from narrow edge 46 of plate 33. The right-angled corner of “L” shaped bar 40 is pivotally anchored to casing 25 by means of axis 41 perpendicular to the bar surface. FIG. 5A represents the “relaxed” mode (i.e., buckle 4 in protracted position), while FIG. 5B is in a “contracted” mode (buckle 4 in retracted position). To understand the action of this embodiment a static description of the “relaxed” mode followed by the “contracted” mode description is herein given. The “relaxed” mode in FIG. 5A, illustrates the electromagnetic motor 32 at a perpendicular position to the base of casing 25, and “L” shaped lever 41 in a perpendicularly positioned to reciprocating rod 32.

The “contracted” mode is shown in FIG. 5B. When reciprocating rod 32 retracts into electromagnetic motor 31, it causes the “L” shaped to rotate around axis 41, such that connection 69 moves toward electromagnetic motor 31 as well as toward the rectangular plate 33. This rotation is allowed due to pivot attachment 99 of electromagnetic motor 31 and pivot attachment 41 of “L” shaped lever bar 40. The other end of the “L” shaped lever bar 41 slides in the upward direction on edge 46 of rectangular plate 33 and at the same time it pushes plate 33 causing it to rotate counterclockwise such that edge 45 and consequently buckle 4 are drawn deeper into casing 25. When reciprocating rod 32 reciprocates its motion, “L” shaped bar 41 returns to its “relaxed” perpendicular position (FIG. 5A) and consequently edge 45, along with buckle 4 are pushed outwardly. Thus, this chain of events leads to an effective intermittent shortening of the limb encircling loop (50) and to an intermittent compression of the underlying muscle enhancing the blood flow.

FIG. 6 illustrates yet another preferred embodiment of the present invention, including means for allowing asymmetrical contraction-relaxation cycle and in particular for allowing fast contractions, followed by much longer periods of relaxation. Such a cyclic pattern is found to have the most beneficial effect for enhancing blood and lymph flow. In accordance with this embodiment, the machinery components responsible for intermittent pulling and releasing strap 1 comprises a motor 121 having a worm shaft 122, a speed reducing gear comprising wheels 124 and 126, coupled to shaft 122, and a disk 128 of irregular perimeter, concentrically mounted on wheel 126. Double-tooth disk 128 is shaped as two identical halves of varying curvature radius, each having a gradual slope at one end and a cusp 129 where the radius changes abruptly from maximum to minimum at its second end, wherein between two ends the radius of curvature is almost constant. The machinery components, including motor and wheels, are accommodated in a central compartment 120 of casing 25. Two side compartments, 110 and 140, accommodate laterally movable strap connectors 105 and 145, respectively. Compartments 110 and 140 are provided with side slits 114 and 141, through which strap 1 can slide in and out. In accordance with the embodiment shown here, strap 1 is retractably mounted at one side of casing 25 (compartment 110) and having its free end provided with a quick male connector for connecting into complementary female connector in compartment 140. This strap fastening arrangement allows for quick and simple adjustment of the strap to the size of the limb and for exerting primary pressure on the muscles. Accordingly, connector 105 includes a vertical rod 102 rotate ably mounted between two horizontal beams 116 and 117, allowing rod 102 to revolve around its axis for rolling or unrolling strap 1. Strap 1 is affixed to rod 102 at one end and is wound around the rod. Rod 102, acting as a spool for strap 1, is provided with a retraction mechanism (not shown). The retraction mechanism can be any spring loaded retracting mechanism or any other retraction mechanism known in the art, such as are used with seat belts, measuring tapes and the like. For example, the retraction mechanism can comprise a spiral leaf spring having one end secured to rod 102 so as to present torque on the rod when strap 1 is withdrawn and to cause the strap to roll back once its free end is released. The upper end of rod 102 terminates with head 115 and a cap 116 of a larger diameter mounted on springs 118. The inner surface of cap 116 fits onto outer surface of head 115, such that when cap 115 is pressed downward, it locks head 115, preventing free rotation of rod 102 and consequently preventing strap 1 from being rolled or unrolled. The second free end of strap 1 terminates with buckle 111 which fits into a complementary accepting recess 142 of connector 145 for allowing quick connection into the second side of casing 25. In the example illustrated here, buckle 111 has an arrow shape while connector 145 has a complementary arrow shape recess 142 provided with slanted protrusions 144 mounted on springs 146. When buckle 111 (duplicated on the right side of FIG. 6 for description sake only) is pushed toward recess 142, protrusions 144 are pressed aside, and then fall behind the arrow head of buckle 111, locking the buckle.

The device is further provided with an on/off switch 130 comprising button head 132, electrical connector 134 made of electric conductive material, and a bottom protrusion 136. When switch 130 is pushed to the left by means of head 132, connector 134 closes the electric circuit (shown in broken line), setting the machinery into action. Simultaneously, protrusion 136 presses cap 116 downward, locking head 115 and preventing rod 102 from turning around its axis, for fixing the available length of strap 1. Button 132 can be further provided with a force regulator for regulating the frequency. Movable connectors 105 and 145 are coupled to the machinery components by means of horizontal rods 106, which extend through openings 103 into central compartment 120 and are in contact with disk 128 perimeter. Horizontal rods 106 terminate with bearings 109 which allow the rods to smoothly slide along disk 128 perimeter as the disk revolves around its axis. Thus, the distance between rods 106, and consequently the periodical change of the circumference of the loop encircling the limb, mimics the outline shape of disk 128. In order to maintain constant contact between bearings 109 and disk 128 and to facilitate fast transition between strap relaxed to contracted position, rods 106 are mounted on biasing springs 108 positioned between walls 105 and are provided with plates 107 perpendicular to the rod axis and pressed against springs 108. Thus, springs 108 bias connectors 105 and 145 in the inward direction toward each other. As disk 128 revolves around its axis, springs 108 are compressed by plates 107 in accordance with disk 128 varying radius. When disk 128 rotates to the point where cusps 129 simultaneously face bearing 109, rods 106 momentarily lose contact with disk 128 and the potential energy stored in springs 105 is released, pushing rods 106 inwardly. This causes a sudden inward pulling of strap 1 by both rods 106, leading to sharp squeezing of the limb muscles. It will be easily realized that the length interval between contracted and released states of the limb encircling loop, and hence the squeezing force exerted on the muscles, is directly proportional to the radius change at cusp 129. Following the sudden strap contraction, the rods are gradually pushed outwardly leading to strap relaxed mode which lasts for substantially half a cycle. Hence, one revolution of disk 128 around its axis results in two fast strap contractions. Typically, the transition from relaxed to contacted position takes about 0.5 seconds, the transition from contracted to relaxed position takes about 5 seconds and the relaxed position is maintained for about 50 seconds. However, it will be easily realized that the perimeter of disk 128 can be shaped such as to obtain any desired contraction-relaxation cyclic pattern. For example, using alternative disk 128 shapes having four cusps rather than two can shorten each cycle by half as well as change the output force of each cycle. It can also be easily realized that disk 128 having a changing radius is energetically efficient allowing the steady build up of energy to be stored in springs 108 during each cycle and to be released in a short burst of high energy output at the end of each cycle. During operation, a low energy output is provided constantly by power source 20 for the operation of motor 121. Constant low energy input is supplied by motor 121 to rotate disk 128 via worm shaft 122 and speed reducing gear wheels 124 and 126, coupled to shaft 122. Rotation of disk 128 coupled to springs 108 via pushing rods 106 provide a steady spring compression as bearing 109 traverses the outer perimeter of disk 128. Energy accumulates in springs 108 in a constant manner until bearings 109 reach cusps 129 when cusps 129 drop from largest diameter to smallest diameter of disk 128 thus allowing pushing rods to quickly slide towards center of disk 128 releasing the energy stored in springs 108 compressing belt 1. It will be easily perceived by persons skilled in the art that this operation is energetically efficient. Furthermore, operating motor 10 at a constant power can be disadvantageous when used with the present invention due to the fact that the force required to compress springs 108 escalates during compression. In order to further enhance the energetic efficiency of the device, the device may be provided with an electric control unit for controlling the voltage applied to the motor for modulating the motor output to match the changing requirements of the system, thus optimizing the motor efficiency. The control unit can be programmed in advance knowing the system requirements during the cyclic course or can operate in accordance with a feedback fed by the motor itself or by another component of the system.

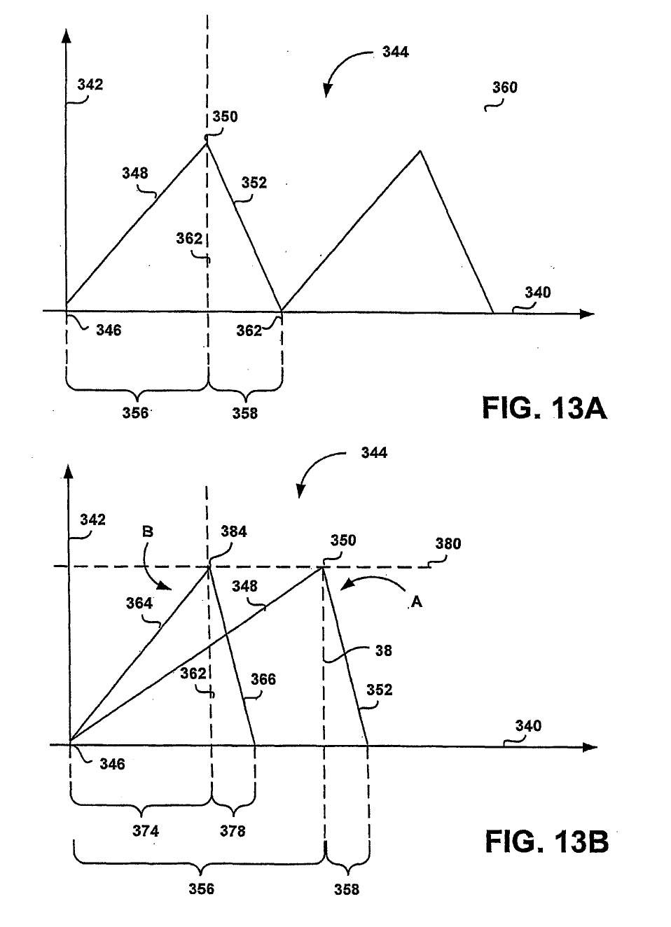

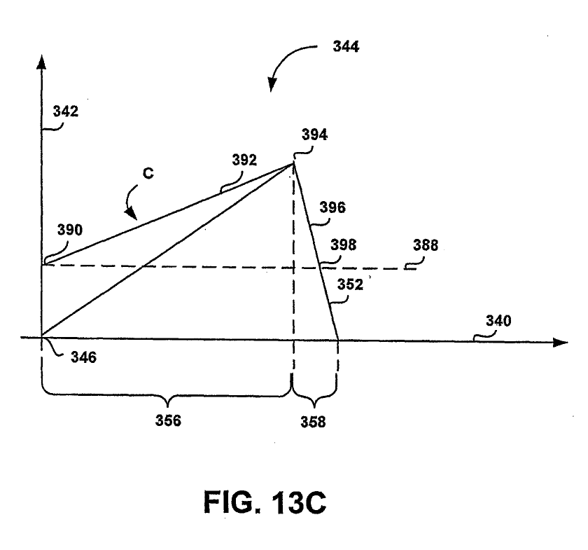

FIG. 13A illustrates one energetic model of the present invention, more specifically a spring energy content graph. The energetic model described hereforth and in FIG. 13A through 13C is a pictorial description of the energy content change in springs 108 of FIG. 6 during periodical operation of the present invention also of FIG. 6 as well as in other figures illustrating the inner machinery of the present invention. Relevant parts described hereforth refer to same parts of the present invention described in FIG. 6. FIG. 13A is a graph describing the energy content of springs 108 versus time during a periodical operation of the present invention. Abscissa 340 depicts a linear flow of time such as in seconds. Other scales can be used such as milliseconds, minutes and the like. Ordinate 342 describes energy content in joules. It should be obvious that Ordinate 342 can describe other elements describing products of energy such as work, pressure, spring length etc. Abscissa 340 and ordinate 342 intersect at point 344 where point 344 is an arbitrary point in time where the energy content of springs 108 is zero and where this point of time is arbitrarily depicted as time of one periodical cycle of operation of the present invention. This point also denotes the time when energy flow through the present invention begins to accumulate via the internal operation of the present invention as further illustrated hereforth.

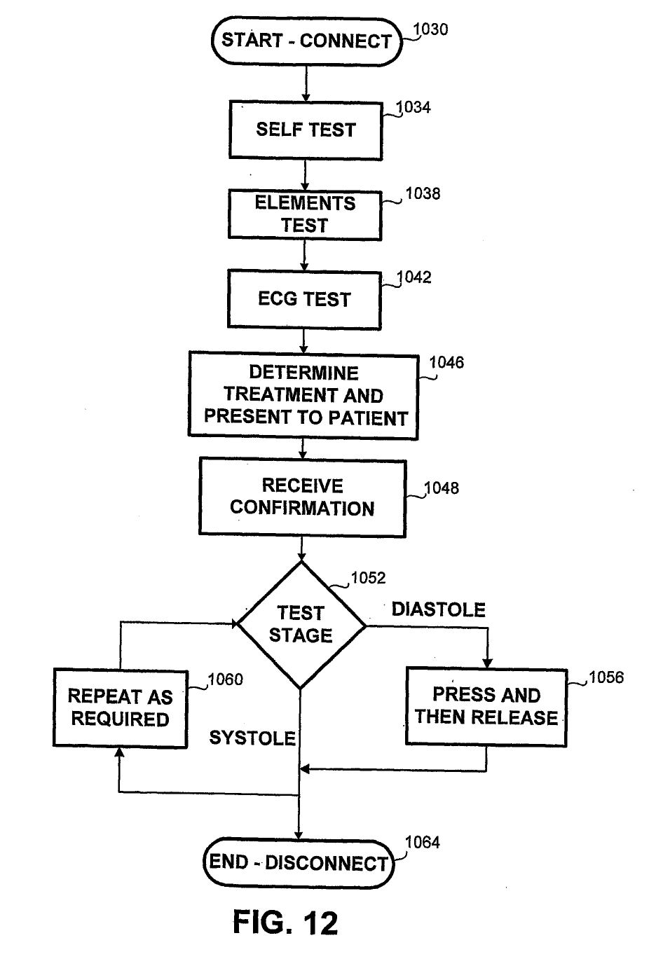

The energy content of springs 108 is now described in conjunction with a partial description of the operation of the present invention with reference to FIG. 6. At point 344 horizontal rods 106 and their corresponding bearings 109 are situated in close proximity of cusps 129 base. At this point springs 108 are in relaxed state where no tension is present on said springs and where the length of said springs is the spring's natural length at zero energy state. As motor 121 is set in motion, constant low energy is produced. This energy transferred constantly through worm shaft 122 as well as speed reducing gear comprising wheels 124 and 126 to a inconstant radius disk 128. Disk 128 is torque to revolve around its axis at a constant speed determined by motor 121 speed output and also determined by shape and size of worm shaft 122 as well as speed reducing gears 124 and 126. As Disk 128 start spinning horizontal rods 106 with their terminal bearings 109 found in constant contact with disk 128 surface starts sliding along disk 128 perimeter. Disk 128 has an inconstant radius such that at each cusp base the smallest diameter exists and at each cusp peak the largest diameter exists. Horizontal rods 106 slide along perimeter of disk 128 from the smallest diameter to the largest one. Such rotational movement of disk 128 imparts linear motion to said horizontal rods 106 pushing them towards side compartments 110 and 140 as diameter of disk 128 increases. Rods 106 via plates 107 which is horizontal to said rods press springs 108 during said motion. As springs 108 shorten, kinetic energy is transferred into spring potential energy. This process of increasing spring potential energy is illustrated in FIG. 13A as line 348. Spring potential energy 348 is accumulated as rods 106 move linearly in the direction side compartment 110 and 140. When rods 106 reach the largest diameter of disk 128 at the peak of cusps 129 springs 108 are at its maximal compression and minimal length. The potential energy stored there at this point of time 362 is maximal and is represented by point 350 on FIG. 13A. The length of time from point 346 to point 350 or the length of time from fully relaxed spring state to fully compressed spring state of springs 108 denoted as time interval 356 in FIG. 12A typically takes 5 seconds but can be in the range of 0.5 to 5 seconds for optimal function of the present invention. At this point in time of the operation of the present invention rods 106 momentarily loss contact with perimeter of disk 128 and briskly move from cusps 129 peak to cusps 129 base towards the center of disk 128. Rapid movement of rods 106 away from springs 108 release compression of plates 107 on springs 108. Springs 108 then return to their natural relaxed state rapidly while releasing their potential spring energy quickly. Peppy energy release 352 of springs 108 is described by line 352 in FIG. 13A. The Potential spring energy is released while spring 108 is lengthening. This produces rapid work utilized for pulling straps 1 towards the center of disk 128 thus enabling the squeezing force of strap 1 on the limb to which the present invention is attached. The peppy energy release time 358 length is typically 0.2 seconds but can be in the range of 0.05 seconds to 0.5 seconds for optimal function of the present invention. Disk 128 continues to revolve around its axis continuously Thus starting another cycle of spring contraction-relaxation. This is denoted by another energy pattern 360. It can be clear to the person skilled in the art that energetic patterns illustrated in FIG. 13A can be changed by changing disk 128 diameter, changing disk 128 revolving speed as well as by adding other elements to the internal machinery which may influence the speed and rate of rods 106 motion through each cycle.

FIG. 13B exemplify the effect of speed change of disk 128 on the energy content graph previously illustrated in FIG. 13A and where like numbers represent like parts. The energy content graph of springs 108 A discussed in FIG. 13A is presented in FIG. 12B where the time interval from spring energy content zero to maximum is represented by the interval 372 and where the peak energy content level of springs 108 is represented by point 350. When spinning speed of disk 128 is increased to twice disk speed discussed in FIG. 13A, represented by graph A, a new spring energy content graph B is created. In this case spring potential energy 348 is accumulated twice the rate as discussed in FIG. 13A and is illustrated by line 364. The maximal energy content 384 of springs 108 is also reached faster. Time interval 374 representing the new time interval from fully relaxed to fully contracted springs 108 also shortens by half, thus time interval 374 is half that of time interval 356. Thus in a different operation mode or in same apparatus having modified internal machinery (not shown) capable of spinning disk 128 faster energy is accumulated within springs 108 faster thus allowing for rapid cycling of the present invention operation. Peppy energy release time 378 is same as peppy energy release time 358 as springs 108 are unchanged and peppy release time 358 and 378 is a function of internal spring properties. It should be clear to the person skilled in the art that different springs with different spring constant (K) can be used as well as internal machinery that regulates springs 108 release time such that peppy energy release time 358 and 378 can be modified thus further modifying the spring energy content graphs. It is clear to the person skilled in the art that a similar but unlike energy content graph (not shown) can be generated by slowing disk 128 spinning speed.

FIG. 13C illustrates yet other spring energy content graphs. Graph A is similar to graph A of FIG. 13B. Two spring energy content graphs are illustrated; spring energy content graphs A which is identical to spring energy content graphs A of FIG. 13A and represent spring energy content related to internal machinery illustrated in FIG. 6 as well as a novel spring energy content graphs C which represent yet another internal machinery characteristics of the present invention discussed hereforth verbally. Spring energy content graph C starts at point 390 on line 388. At this point springs 108 are not fully relaxed where their energy content at the beginning of each operation cycle is not zero. This means that some mechanical or other element such as a stopper (not shown in FIG. 6) is preventing springs 108 from stretching to their fully relaxed state. Spring potential energy accumulation 392 is represented in FIG. 13C by a non linear line starting at point 390 and ending in point 394. The non linearity of line 392 represents a non-linear diameter change of disk (not shown in FIG. 6). Such non-linear diameter disk can alter the operational mode of the present apparatus to suit the specific need of each person using the device. Other elements within the internal machinery of the present invention may also contribute to the creation of such spring potential energy accumulation 392 such as having rod 106 being of an elastic material, having rods 106 being assembled from two stiff rods interspersed by a spring and the like. It is clear from the illustration that peak spring energy of both springs Peppy energy release 396 is similar in slope to peppy energy release 352 indicating springs of same internal constant. Peppy energy release 396 however ends in point 398 where not all the potential energy stored within springs 108 is released as work. This may be achieved by having a stopper (not shown) or other element (as illustrated hereforth in other embodiments of the present invention) with internal machinery of the present invention known in the art for achieving such result. It is clear to the person skilled in the art that only partial springs functionality is achieved with spring energy content graph C such that spring of said graph C stretch and relax at a fraction of their capability. Such a design may be advantageous for certain modes of operation of the present invention.

FIG. 13A through 13C illustrate different energy content graphs representing in actuality different stretching and relaxation times and strength of strap 1 of FIG. 2A thus attaining the purpose of suiting the present invention to aid in the flow of blood and lymph in limbs of persons using the present invention. Each condition requires a different operational mode for best results that are achieved by using said alternate internal machinery alterations. For example, in patients with diabetes mellitus suffering from related circulation disturbances a fast release of strap 1 of FIG. 1A is advantageous for achievement of best circulation pattern. This is achieved by using disk 128 of FIG. 6 having smaller diameters thus reducing relaxation time. This can also be achieved by using different springs 108 also of FIG. 6 having properties allowing fast contraction. This relatively fast relaxation of strap 1 creates a vacuum like effect within the tissue which is optimal for blood flow enhancement in said patients. It is obvious that pressure gradients and flow volume within vessels of person using the present invention are different from ones generated by Intermittent Pneumatic Contraction (IPC) devices used for the same purpose due to the different machinery and material used. It is also obvious to the person skilled in the art that changing parameters of stretch and relaxation patterns as well as energetic patterns stemming from the material and parameters change stated above is relatively easily achieved and performed.

The present device also uses the human tissue (leg matrix) of the user of the present invention as a recoil spring. During the fast squeeze of the human tissue of the user of the present invention some potential energy is stored in tensile elements of the tissue. When relaxation period arrives this kinetic energy is transferred via relaxing tissue to the relaxing strap 1 and thereby aiding indirectly the action of motor 121 of FIG. 6. This allows the usage of smaller and less powerful motor for the achievement of the same results. In the examples discussed above it can be seen that the present invention is also very efficient apparatus for the purpose of blood flow and lymph flow enhancement.

Furthermore, operating a motor at a constant power can be disadvantageous when used with the present invention due to the fact that the force required to compress a spring escalates during compression. In order to further enhance the energetic efficiency of the device, the device may be provided with an electric control unit for controlling the voltage applied to the motor modulating the motor output to match the changing requirements of the system, thus optimizing the motor efficiency. The control unit may be programmed in advance, knowing the system requirements during the cyclic course, or can operate in accordance with a feedback fed by the motor itself or by another component of the system.

A different embodiment of the present invention in which box assembly 2 is the active intermittent compressing part is depicted in FIG. 2B. According to this embodiment, assembly box 2 further comprises a compressing plate 3 lying substantially parallel to casing 25 at a predetermined distance from its surface. According to this embodiment, the assembly 2, more specifically said compressing plate 3 is pressed against the muscle and intermittently extend and retracts from casing 25 thus producing intermittent compression of the calf muscle. According to this embodiment strap 1 is connected to casing 2 by two fixed slited latches, such that at least one end of strap 1 is threaded through one of latches 68 and is folded onto itself to allow comfortable fitting, as described in conjunction to FIG. 2B. An on/off switch 6, a power regulator 5 and a rate regulator 7 are located at the top of the device in the same fashion as in FIG. 2B.

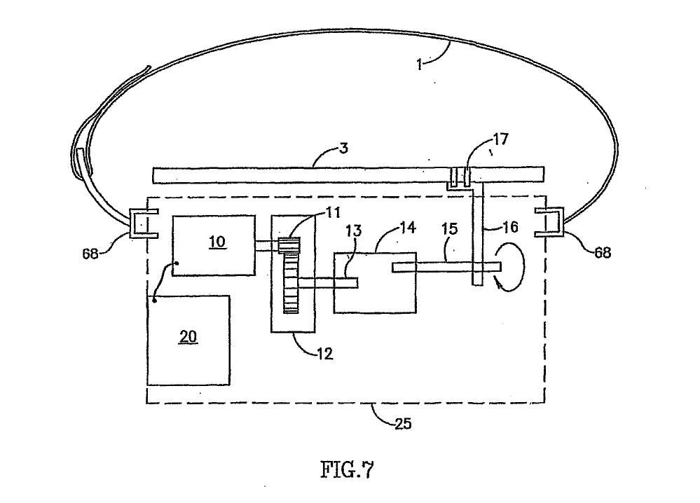

A top view of a machinery embodiment in accordance with the device embodiment of FIG. 2B is shown in FIG. 7. A power source 20 powers an electrical motor 10 that has a centrally located shaft 11. Said centrally located shaft 11 is coupled to a velocity reduction gear 12 which reduces the spinning velocity of the rod 11 and increases the power output. Reduction gear 12 has a centrally located rod 13 that is connected to drum 14 that has an eccentric located rod 15. The eccentric located rod 15 is connected perpendicularly to the longer arm of a motion transfer L-shaped bar 16, wherein the shorter arm of said L-shaped bar 16 is connected to compressing plate 3 by connection means 17. Connection means 17 may be for example bolts, pins, screws and the like. Electrical motor 10 converts electrical energy into kinetic energy stored in the spinning of the centrally located rod 11. The kinetic energy stored in the spinning of the said centrally located rod 11 is converted into power by the said velocity reduction gear 12. The power stored in the said centrally located rod 13 connected to the said velocity reduction gear 12 is converted to the rotation of the said drum 14 which has the said fitted eccentrically located rod 15. The circular motion of the said eccentrically located rod 15 is transferred to the extension and retraction of the said compressing plate 3 via the said motion transfer rod 16 and connection means 17. According to this arrangement, the circular motion of the eccentrically located rod 15 is transferred into periodical motion of plate 3. Said periodical motion of plate 3 is a combination of a first periodic motion in the extension-retraction direction (i.e., increasing and decreasing the distance between plate 3 and casing 25) as well as a second periodic motion which is perpendicular to said first periodic motion. (In accordance with FIG. 6, this second periodic motion is in a direction perpendicular to the drawing surface). Thus, further to the obvious effect of applying intermittent compression on the limb by the extension-retraction motion of plate 3, the present embodiment also imparts the device a “massage-like” effect, thus enhancing the squeezing efficacy. It will be easily realized by persons skilled in the art that the embodiments described in FIGS. 3-7 are only examples and that different features described separately in conjunction with a particular embodiment, can be combined in the design of a device of the present invention. For example, a retractable strap feature as illustrated in FIG. 6 can be combined with any of the other embodiments described herein before and after. Much the same, an asymmetrical component such as disk 128 of FIG. 6 can be added to any of the other embodiments for allowing a particular pattern of a contraction-relaxation cycle.

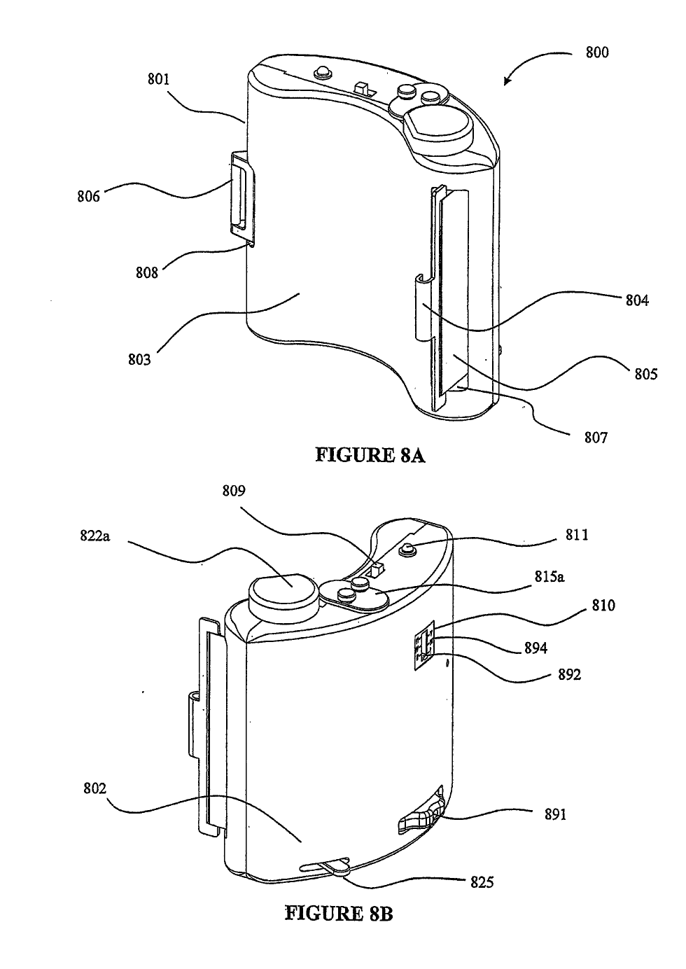

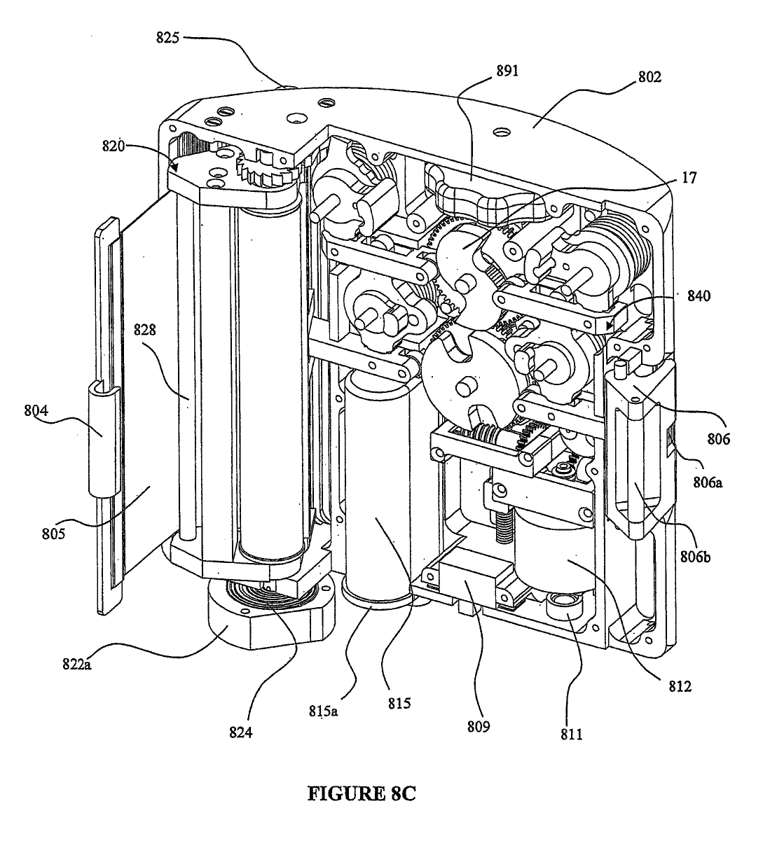

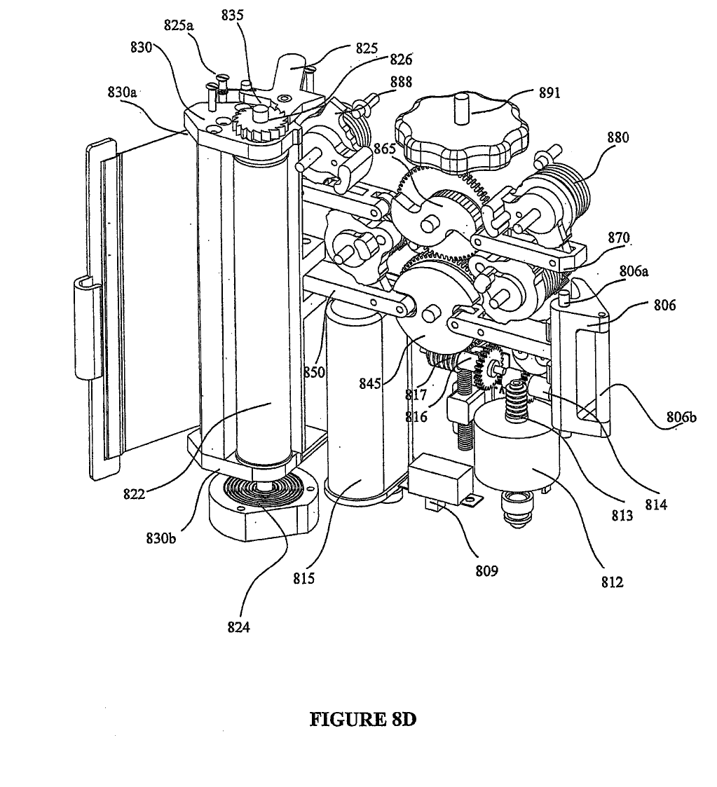

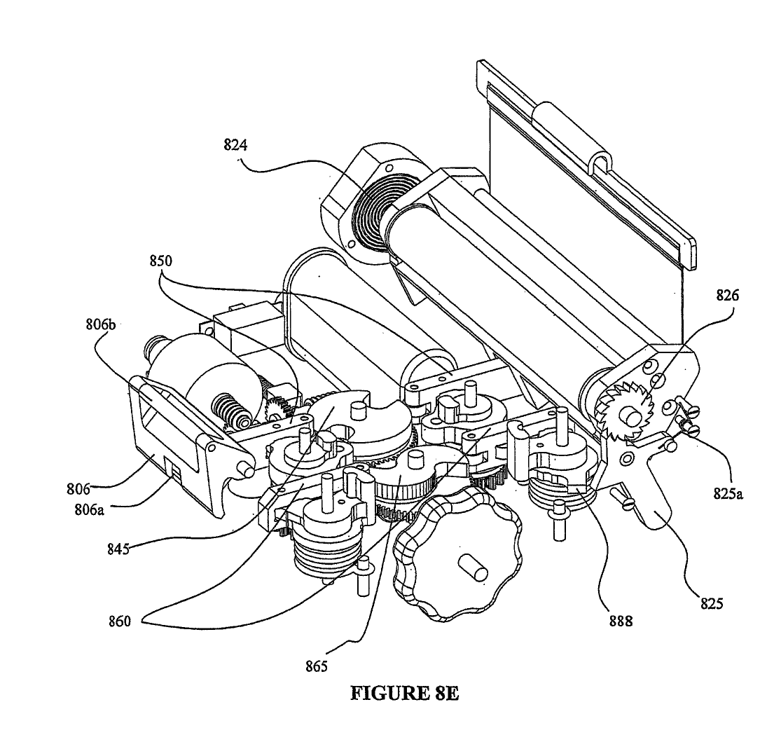

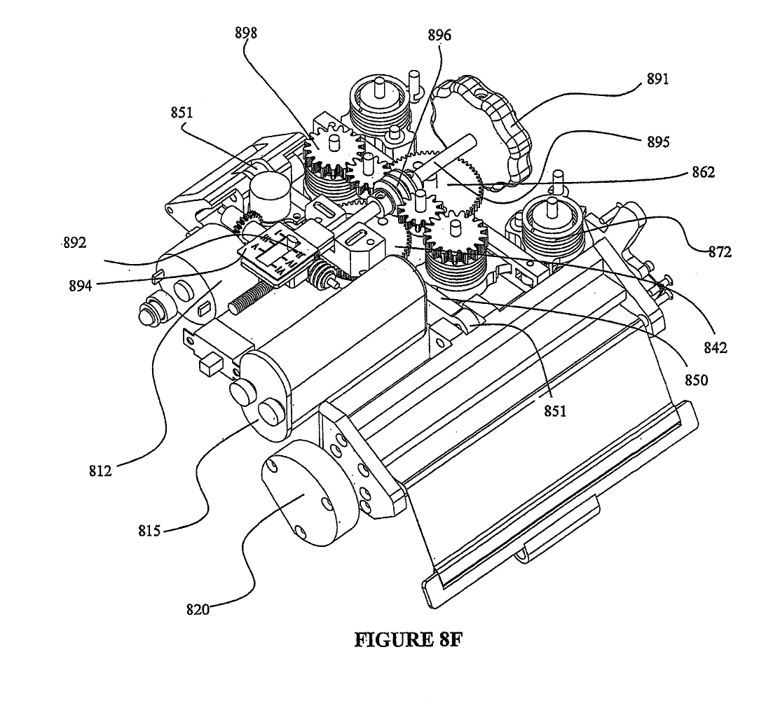

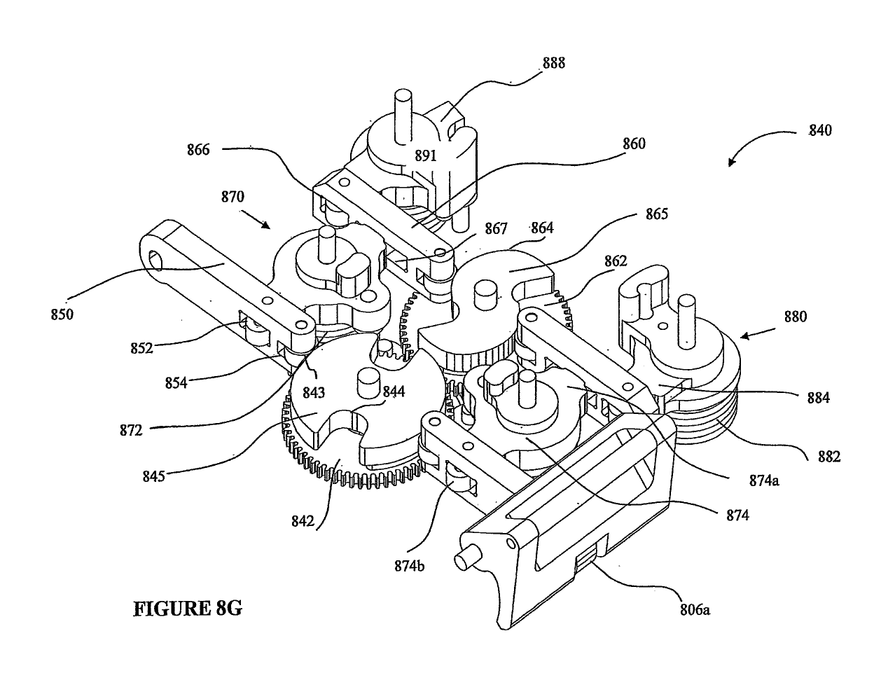

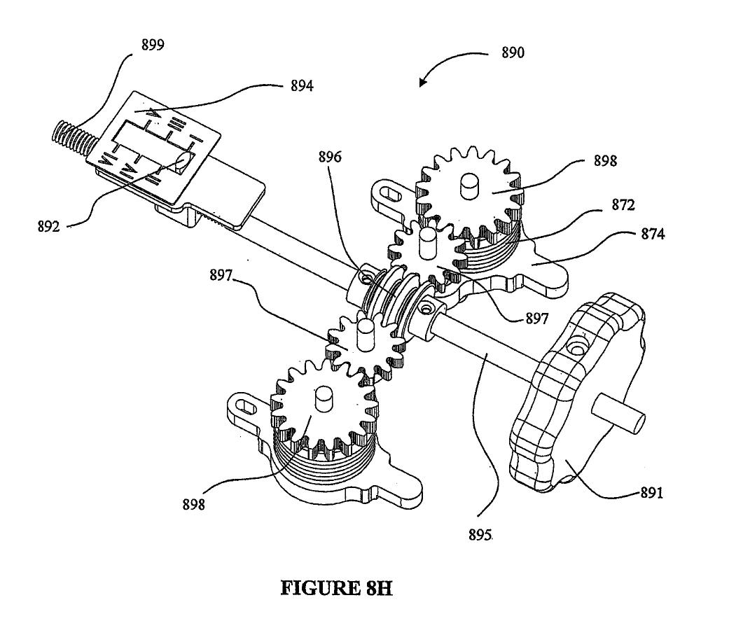

Referring now to FIGS. 8, there is illustrated a further embodiment of the present invention with an enhanced contraction—relaxation internal machinery, which provides reverse propulsion mechanism. In particular, the present embodiment allows for a fast transition from relaxed to contracted state, as well as, from contracted to relaxed state. A fast transition from contracted to relaxed state, which induces sudden expansion of blood vessels, is of particular benefit in some circulation disorders, such as for example those resulting from diabetes mellitus, congestive heart disease and the like. Furthermore, the present embodiment is highly efficient in terms of power consumption as it utilizes a relatively low power motor to charge potential energy into springs for enabling fast high power transitions.

FIGS. 8A and 8B are perspective rear and frontal views, respectively, of the reverse propulsion device, generally designated 800. Device 800 is a flask-like casing box 801, similar in shape to casing 25 of FIG. 2A, comprising a frontal cover 802 and a back cover 803. Device 800 can be housed in various shape casings. A strap 805 retractably wound about strap roller 822 encased inside the box (as best seen in FIG. 8C) and terminating with a strap hook 804, is drawn through opening 807 to be engaged with rotating buckle 806, protruding from opening 808, for encircling the user limb (not shown). A strap roller unlock latch 825 extending from frontal cover 802 allows the user to pull the strap before use in order to put the device on the limb and to disconnect the device after use. During operation, roller strap 825 is locked automatically before transition from relaxed to contracted state and is unlocked automatically after transition from contracted to relaxed state, as will explained below. A spring force adjustor wheel 891, coupled to force adjusting mechanism 890 (shown in detail in FIG. 8F) allows for adjusting the force applied on the limb in accordance with the user needs prior to operation. The value of the force is indicated by a pointer 892 on force scale 894 through transparent window 810. Also shown on the top of casing 801 are strap roller cover 822a, battery cover 815a, an on/off switch 809 and a LED indicator 811 for indicating low battery power.

An overall view of the internal components of device 800 is given at different perspective views in FIG. 8C through 8F. Throughout FIGS. 8A to 8H like numerals refer to like elements.

Deice 800 is driven by motor 812 powered via on/off switch 809 by batteries accommodated in battery compartment 815. Preferably the motor 812 is a small light weight motor powered by one or more AA batteries of 1.2-1.5V. During operation motor 812 operates continuously. The rotational motion of motor worm shaft 813 is transferred via transmission gear comprising a first and second speed reducing gears 814 and 816 to gear 842 of the reverse propulsion assembly, generally designated 840, via worm 817 of gear 816 (best seen in FIG. 8E). The reverse repulsion mechanism 840 is responsible for the contraction-relaxation cycle of strap 805 by intermittently pulling linear arms 850 toward and away from each other, thereby rotating buckle 806 and strap roller arm 830 around axes 806a and 835 respectively, to increase the tension of strap 805 when arms 850 are pulled inwardly and to release the tension when the arms are pulled outwardly. The internal components of device 800 also include strap roller assembly 820 and force adjustment assembly 890. For clarity sake, the following description will be divided into separate descriptions of the roller strap assembly 820, the reverse propulsion mechanism assembly 840 and the force adjustment assembly 890. However, it should be understood that the division is artificial as the different assemblies are coupled to each other and share common elements. Roller assembly 820 includes a strap roller 822 mounted within strap roller arm 830 and a roller lock/unlock latch 825. Strap roll 822 is having a central axis 835 rotatably mounted between two horizontal plates 832a and 832b of roller arm 830 and extending there from. One end of axis 835 is connected to winding spiral spring 824 for providing a retracting force on strap 805. The retracting force on strap 805 can be chosen to provide a constant low pressure on the limb during the relaxation phase. This low pressure, referred to as ‘pretension’ is preferably in the range of 5-15 mmHg. The other end of axis 835 is provided with ratchet wheel 826 fixedly mounted thereon. Lock/unlock latch 825, biased by spring 825a toward ratchet wheel 826, is configured to engage with ratchet wheel 826 for preventing free rotation of axis 835 when engaged, as can be best seen in FIG. 8E, hence disabling spring 824 and preventing strap 805 from rolling/unrolling about roller 822. Thus, when as latch 825 and ratchet 826 are engaged, the total available length of strap 805 is maintained constant. Roller arm 830 further comprises a fixed rod 828, extending between the outward comers of plates 830a and 830b, around which strap 805 is passed. Roller arm 830 is rotatably mounted around axis 835 and is pivotally connected to linear arm 850 by hinge 851 provided at the distal end of arm 850 (best seen in FIG. 8F). It can be seen that when roller arm 830 is pulled inwardly by arm 850, arm 830 rotates clockwise (CW) around axis 835 to move rod 828 toward the front cover 802 and away from the limb. It can be also seen that rod 806b undergoes a similar movement (but in a mirror image fashion) when rotating buckle 806, rotatably mounted around axis 806a and pivotally connected by means of hinge 851 to corresponding arm 850, is pulled inwardly. Thus, pulling arms 850 inwardly, result in increasing tension in the strap. If at this time, latch 825 and 826 are engaged, to maintain the available length of the strap constant, the tension in the strap cannot be released and the effective length of the strap shortens. The positional shift of roller arm 830 and buckle 806 between loose to contracted strap states can be best understood by comparing FIG. 8C (loose state) and 8D (contracted state). Strap roller assembly 820 is coupled to reverse propulsion mechanism 840 not only by linear arm 950 but also by means of wing 888 which disengages latch 825 from ratchet wheel 826 during relaxation phase, as will be explained below, to allow continuous adjustment of strap 805 length to the user limb. The continuous adjustment of the strap allows for continuous operation of the device for prolong time period with no need to stop operation to readjust the strap.

Turning now to FIG. 8G, Reverse propulsion mechanism assembly 840 is continuously driven by motor 812 by means of gear 842, meshed with worm gear 817, as explained above. Assembly 840 includes a strap contraction timing disk 845 concentrically mounted on gear 842 interposed between two contracting arms 850 and a strap release S-shaped disk 865 fixedly mounted on gear 862 interposed between two releasing arms 860. Gears 842 and 846 are meshed with each other resulting in opposite rotation of disk 845 and 865. Disk 845 perimeter consists of two arcs 843 of constant radius interrupted by two opposite recesses 844 of smaller radius. S-shaped disk 865 is shaped to have two arcs 864 of increasing radius ending by a cusp where the radius abruptly changes from maximum to minimum. Assembly 840 further comprises two sets of spring assemblies, contraction spring assemblies 870 and release spring assemblies 880. Contraction spring assembly 870 includes a spring 872 and a rotating timing arm 874, having a distal end 874a and a proximal end 874b, mounted thereon. Release spring assembly 880 includes a spring 882 and a rotatable arm 964 mounted thereon. Spring assembly 880 proximal to roller assembly 820 is further provided with wing 888 for allowing pushing latch 825 away from ratchet wheel 826 during relaxation phase for unlocking axis 835. The springs and arms are configured such that clockwise rotation of the arms of the spring assemblies on the left side of FIG. 8G and counterclockwise rotation of the arms on the right side of FIG. 8G load the corresponding springs. Contracting arms 850 are each having an aperture 852 for receiving the proximal end 874b of timing arm 874 of contracting spring assembly 870 and are each provided with bearing 854 at the inner end for allowing the arms to slide along the perimeter of disk 845. It can be easily seen that as long as arms 850 are in contact with arcs 843 of disk 845 the strap is in relaxed position and that when the arms are moving into recesses 844, the strap is in the contracted position. Releasing arms 860 are each having a back aperture 866 for receiving rotating arm 884 of release spring assembly 880 and a middle wider aperture 867 for receiving the distal end 874a of timing arms 874 of contracting spring assembly 870, such that timing arms 874 couple between release arm 860 and contraction arms 850. The inner ends of arms 860 are provided with bearing 868 for allowing sliding along the perimeter of disk 865. Strap contraction springs 872 are biased to push arms 850 via arm 874 toward contraction timing disk 845. Release springs 882 are biased to push release arms 860 via arm 884 inwardly such that bearings 868 are constantly pressed against S-shaped disk 865 following the disk contour. Springs 872 and 882 are selected such that the torque of spring 882 is always higher that of spring 872 so that during all stages of operation, the force exerted on arm 850 by spring 882 (via arms 884 and 874) overcomes the opposite force exerted on the arm by spring 872. This force relation between the springs combined with the positional relation between disks 845 and 865 as they revolve around their centers allow for fast extraction of arms 850 from recesses 844, as will explained in more detail below.

Turning now to the action description of the present embodiment, it will be easily realized by the person skilled in the art that both sides of the present invention work in unity and thus should be viewed. It will be also understood that although the following description is given in a serial fashion, some of the actions described hereforth occur simultaneously and are described in a fractionated fashion for the sake of clarity only.

During operation, gear disk 845 and 865 are continuously rotating counterclockwise and clockwise, respectively, as indicated by the arrows. As disks 845 and 865 revolve each around its center, release arms 960 follow the perimeter of S-shaped disk 865 while contraction arms 850 follow the perimeter of disk 845. Disks 845 and 865 are configured such that as arms 860 follow increasing-radius arcs 884 of disk 865, arms 850 are in contact with constant-radius arcs 843 of disk 845. Thus, as long as recesses 844 are not directed toward arms 850, arms 850 slide against disk 845 and the strap is in the relaxed state while at the same time arms 860 are pushed outwardly by the increasing radius of disk 865 against springs 882 to load springs 882 and simultaneously to release the distal end 874a of arm 870 to freely move within aperture 867. Also during relaxation phase, wing 825 of left arm 880 pushes latch 825 away from ratchet wheel 826, enabling free rotation of roller 822. Thus the only strain in strap 805 during relaxation phase is due to the low force of retracting spring 824 and the available length of the strap may adjusts itself to changes in the limb circumference. However, as arms 860 are pushed outwardly, wing 888 of left arm 880 rotates inwardly away from latchet 825 although still in contact therewith. Wing 888 is configured to lose contact with latch 810 shortly before recesses 884 arrived at a position opposite arms 850, thereby latch 825 engages ratchet wheel 826 to lock roller 822 and to maintain the available length of strap 805 constant. When recesses 844 reach a position opposite arms 850, the arms abruptly fall into the recesses due to the force exerted by spring 872 via arm 870, resulting in abrupt rotation of buckle 806 and roller arm 830 and consequently with fast contraction of the effective length of strap 805 to apply a sudden squeezing of the limb. At this point, disk 865 is positioned such that arms 860 are very close to but not yet reached the disk cusp and springs 882 are loaded close to maximum. As the disks continue to revolve around their centers, arms 860 slide beyond the cusp of disk 865 and fall inwardly due to the force exerted by spring 882. At the same time, arms 850 are abruptly extracted outwardly from recesses 844 by the sudden force exerted in the inward direction on distal end 874a of arm 870 which overcomes the opposite force exerted on proximal end 874b by spring 872, resulting in relaxation of the strap. Thus, timing arms 874 transmit the abrupt inward motion of releasing arms 860 to an abrupt outward motion of arms 850. At this stage, as wing 888 is still turned away from latch 825, latch 825 is still engaged with wheel 826 to maintain the available length of strap 805 constant. As the disks further revolve, arms 860 are pushed outwardly by increasing-radius arcs 864 of disk 865 to release distal ends 974a of arms 874 such that the only force exerted on arms 850 is that of spring 872 and consequently contraction arms 850 are pushed inwardly to be brought again into contacts with arcs 843 of disk 845, wing 888 is brought into contact with latch 825 to unlock roller 822, and the cycle starts all Over again.

It will be realized by persons skilled in the art that although mechanism 800 as illustrated in FIGS. 8 is configured to provide fast contraction followed shortly by fast relaxation, the embodiment can be configured such as to allow time delay between relaxation and contraction. This can be achieved, for example, by enlarging recesses 844 and by coinciding the cusps of disks 865 to arrive opposite arms 860 shortly before arms 850 reach the recess ending. Alternatively or additionally, disk 845 can be mounted on gear 842 in a way which allows a limited relative rotation between disk and gear, for example by mounting disk 845 in arched grooves engraved in upper surface of gear 842. This will allow for disk 845 to remain locked by arms 850 while disk 842 keeps rotating, until by appropriate selection of disk 865, arms 850 are extracted from recesses 814 to allow further rotation of disk 812. A limited relative rotation between disk 845 and gear 843 also allows for recoil of disk 845 when arms 850 fall into recesses 844, facilitation smooth transition by avoiding mechanical stress.

From the above description it should be realized that the squeezing force applied to the limb is directly proportional to the potential energy of springs 872 right before arms 950 fall into recesses 844 which in turn is determined by the initial energy of the spring. Force adjusting assembly 890, shown in detail in FIG. 8F, allows for adjusting the force of springs 872 by winding the springs by means of tooth wheels 898 connected to the second end of spring 872 wherein the first end is connected to arm 970. Assembly 890 comprises an axis 895 provided at one end with wheel 891 protruding from frontal cover 802, having a concentrically worm gear 896 mounted thereon and ending with worm 999. Wheels 898 are coupled to worm gear 896 by means connecting tooth wheels 897 such that turning wheel 891 in one direction winds springs 872 to increase the spring force while turning the wheel in the opposite direction will decrease the spring force. The force of spring 972 is indicated by movable pointer 892 mounted on worm 899 to move along the worm upon turning of axis 895, through scale 894 fixedly mounted to axis 894. The adjustment of the force by wheel 891 is performed by the user prior to operation of the device. Typically, the force of spring 972 varies in the range of 2 to 10 Kg, for applying a pressure in the range of 30-90 mmHg. It will be realized that different users requires different force to obtain the same pressure since the pressure applies on the limb depends on the area of the strap encircling the limb which in turn is determined by the circumference of the limb at the locale where the device is applied. Thus, users having larger limb circumference will need the device to operate at higher force than those having smaller limbs. Furthermore, the optimal pressure is varied from one user to another. Accordingly, device 900 may be provided with a correlation table giving correlation ratios between the force read in scale 894 and the pressure obtained as function of the limb circumference.

For complete understanding of the operation of the present embodiment it must be clear to the viewer the two sets of spring assemblies, namely contraction spring assembly 870 and release spring assembly 880, provide forces that allow fast contraction as well as fast relaxation of strap 805. In this respect, it is important to note that in persons having certain medical conditions such as diabetes mellitus blood flow, enhanced flow is directly proportional to the relaxation time of the strap. The mechanism of the present embodiment provides for a fast relaxation of the strap, thus enhancing blood and lymph circulation in theses conditions considerably.

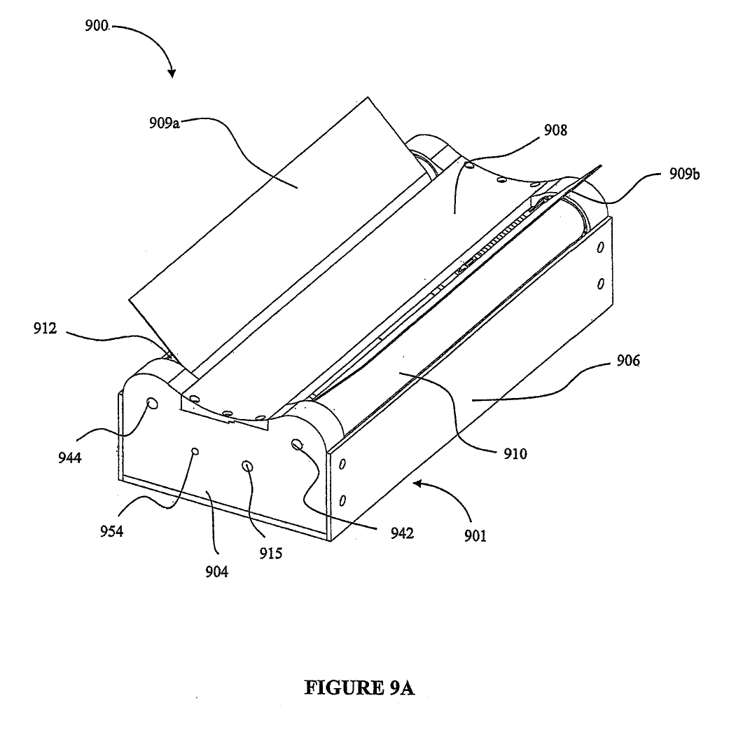

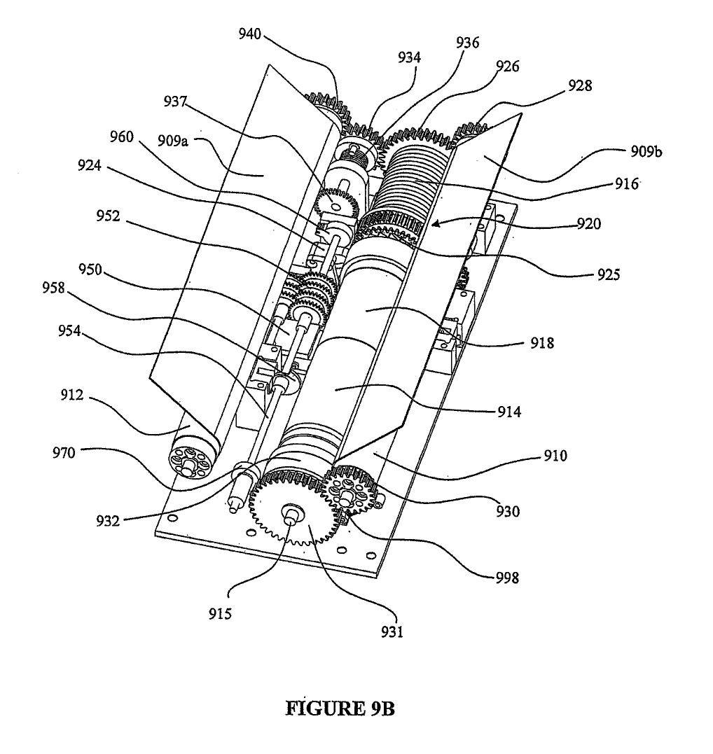

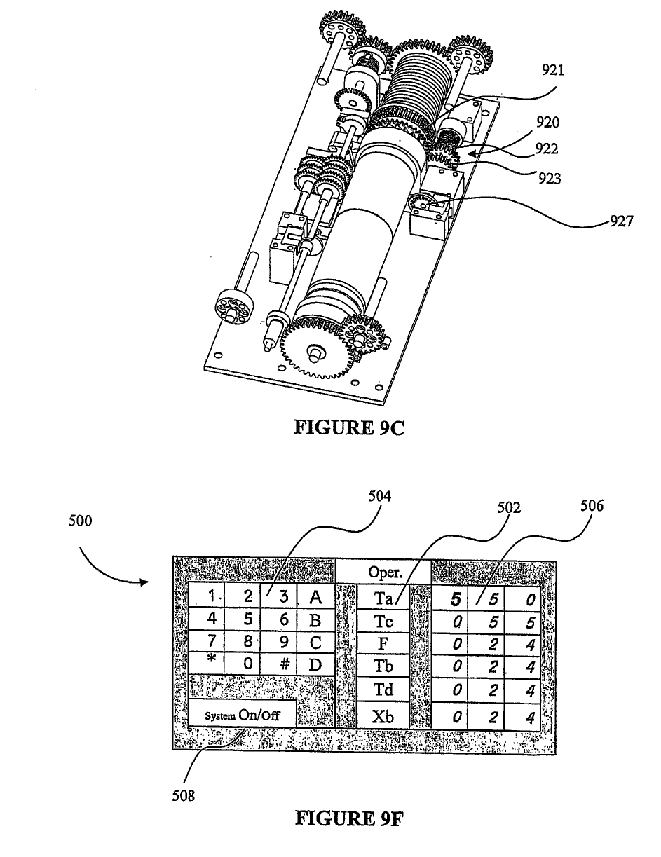

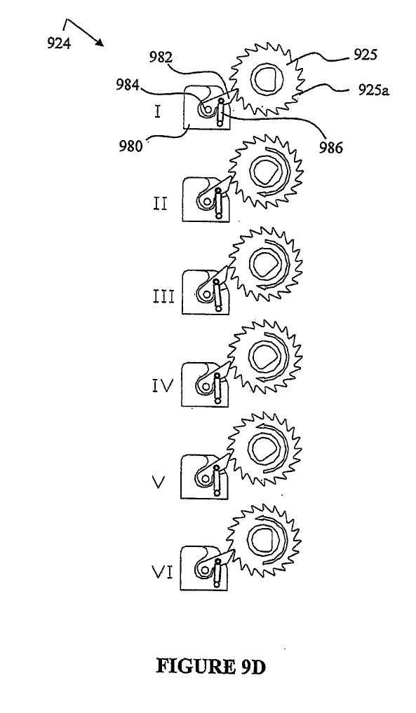

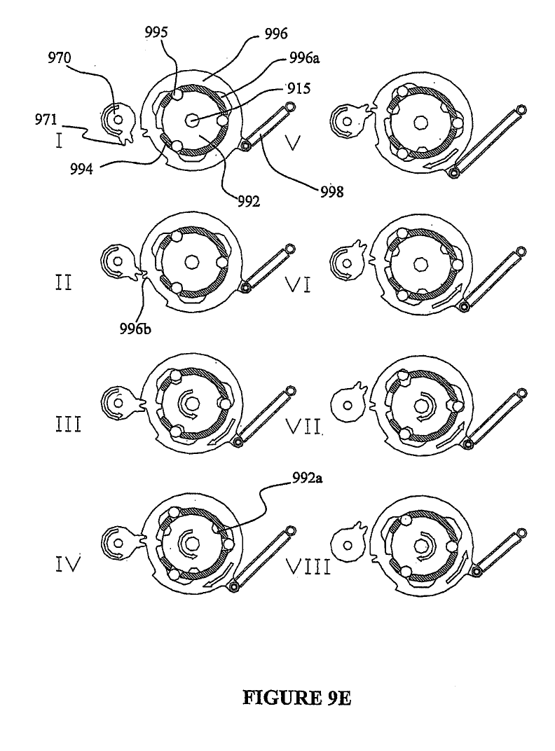

Turning now to FIGS. 9, an alternative embodiment is described where rotational motion of coiling springs, gears and rollers results in intermittent fast transitions between relaxed and contracted states of a strap encircling a user limb. The embodiment described herein, generally designated 900, comprises an external case illustrated in FIG. 9A and internal machinery illustrated in detail in FIGS. 9B through 9F.

Referring to FIG. 9A, case 901 is a substantially elongated rectangular box made of light and strong material such as a composite metal, strong plastic and the like. Box 901 comprises a substantially rectangular flat base plate 902 on which the internal machinery is mounted and two pairs of side plates 904 and 906. Two elongated rollers, right roller 910 and left roller 912 are rotatably mounted around axes 942 and 944, respectively, extending the length of the box between opposite plates 904. Two straps 909a and 909b wrapped around rollers 910 and 912, respectively, are connected to each other to form a closed loop around the user limb such that when the rollers spin in opposite directions the effective length of the combined strap is shortened or lengthened depending on the rollers spin direction. Straps 909a and 909b may be fastened to each other by various fasteners known in the art such as Velcro strips, various buckles and the like. Alternatively, device 900 can be provided with relatively short free ends of straps 909a and 909b to be fastened to a tubular sock-like garment worn on the limb prior to application of the device. Preferably, at least one elastic element in incorporate into at least one of straps 909 for providing a limited elasticity to the strap. A plate 908, positioned between rollers 910 and 912, covers the middle section of case 901, leaving gaps between plate and rollers to allow revolutions of strap 909 around the rollers. Plate 908 is a curved plate designed to fit snugly over a limb. Plates 902, 904, 906 and 908 are affixed to each other by any means known in the art such as glue, bolts and the like. Embodiment 900 is attached to a person's limb (not shown) via strap 909 with plate 908 being in contact with the limb in a similar fashion as in anterior box embodiment of FIG. 1A.

Referring now to FIG. 9B and 9D, the internal machinery includes a main motor 914, a planetary transmission 918 and a mainspring 916 coupled to planetary transmission 918 via mainspring clutch 920. Helical spring 916 is fixedly secured between top mainspring gear 926 and clutch gear 921 of clutch 920. Clutch 920 includes an external clutch spring 922 coupled to gear 921 via gearing 923 such that the torque of clutch spring 922 is proportional to the torque of mainspring 916. A ratchet mechanism 924, the details of which are shown in FIG. 9E, prevents via ratchet wheel 925 reverse rotation of gear 921 and consequently reloading of spring 916 as long as clutch 920 is locked. The top mainspring gear 926 is meshed on one side with right roller top gear 928 and on the other side with connect gear 934 which in turn is meshed with left roller top gear 940, coupling between the mainspring 916 and rollers 910 and 912 such that rotation of gear 926 results in simultaneous and opposite rotation of rollers 910 and 912. A strap return spring 936 of a lower spring constant than that of mainspring 916, is connected to gear 934. Helical spring 936 is configured to be loaded in the opposite direction to that of mainspring 916. Turning now to the bottom part of FIGS. 9B-9D, a strap contraction clutch 932 is coupled to right roller bottom gear 930 via strap contraction clutch gear 931. Clutch 932 locks/unlocks gear 931 and consequently locks/unlocks rollers 910 and 912 via gears 928, 926, 934 and 940. The machinery further comprises a timing assembly comprising a timing motor 950 coupled via transmission 952 to timing shaft 954. Two offset double-tooth cam release disks 960 and 970 are mounted on shaft 954 in alignment with main spring clutch 920 and strap stretching clutch 932, respectively, constructed to engage therewith for unlocking corresponding clutch. In accordance with the embodiment shown here, the mechanism further comprises a main spring encoder 927 mounted on the axis of spring 922 of clutch 920 for reading mainspring 916 torque, a timing shaft encoder 958 mounted on timing shaft 946 for reading the angular positioning of disks 960 and 970 and a strap length encoder 937 mounted on the axis of gear 934 for reading the strap effective length and velocity during transitions. The readings of encoders 927, 958 and 937 are fed into a microprocessor (not shown) which also controls motors 914 and 954.

The following description is divided into three phases of the internal mechanism action. The first phase is the loading phase during which mainspring 916 is loaded and the effective length of the strap remains constant in the relaxed state. The second phase is the strap shortening phase during which abrupt squeezing forces are applied to the encircled limb followed by a predetermined period of time during which the effective length of the strap remains in the contracted state until the third phase is actuated. The third phase is the relaxation phase where the strap effective length returns to its relaxation length by fast transition. The three phases follow each other in time, providing intermittent fast transitions from relaxed to contracted state and vice versa.

Loading phase. During loading phase, strap release clutch 920 and 932 are locked. Loading phase starts with the effective length of the strap being in the relaxed state, by activating motor 914. With clutches 920 and 932 locked, motor 914 via transmission 918 loads mainspring 916 by actuating rotational motion of the proximal end of the spring (proximal to motor 914. Main motor 914 may operate at constant power or alternatively motor 814 may operate with variable output such that as the torque of spring 916 increases so does motor 914 power for maintaining constant rate of spring loading rate. Planetary transmission 918, the internal construction of which is not shown, may be any known in the art planetary transmission for allowing angular speed reducing along a rotation axis. As already mentioned, during the loading phase strap contracting clutch 932 is locked, preventing rotational motion of any of gears 930, 928, 926, 934 and 940. Thus, although the torque built up in mainspring 916 is transferred via gear 826 to upper rollers gears 828 and 840, rollers 910 and 912 cannot rotate and consequently the effective length of the strap remains constant. The torque built up in mainspring 916 is monitored by encoder 927. When mainspring 916 reaches a predetermined value, motor 914 is turned off thereby halting further loading of the spring. At this stage, when no voltage is applied to motor 914, locking ratchet 924 prevents rotation of gear 921 in the reverse direction, hence prevents mainspring 916 from relaxing and maintains the mainspring torque.

Shortening phase. During shortening phase, clutch 920 remains locked. The transition from relaxed to contracted state is controlled by the timing mechanism via release disk 970 configured to unlock strap contracting clutch 932 upon engagement therewith. The shortening phase is effectuated by turning on motor 950 whereupon rotational motion is transferred via transmission 948 to timing shaft 954. Consequently, disk 970 rotates to a position where the disk teeth engage with corresponding teeth on external cylinder of clutch 932 to unlock the two parts of the clutch, as is illustrated in detail in FIG. 9E, and to allow disk 931 to freely rotate around its axis. Unlocking disk 931 unlocks disks 928, 926, 934 and 940 as well. Thus, unlocking clutch 932 while clutch 920 is still locked for preventing rotational motion of disk 921, immediately results in partial release of the system strain through clockwise rotational movement of mainspring gear 926 and consequently in counterclockwise rotation of right roller 910 and clockwise rotation of left roller 912. This results in abrupt shortening of the effective length of the strap and high power squeezing forces on the limb, until no further shortening is possible due to the limb resistance. At the same time that mainspring 916 is partly unloaded, return spring 936 is loaded by the rotational motion of connect gear 934. Thus, the release of clutch 932 brings to both strap 909 shortening and return spring 936 loading. The rotation of connecting gear 934, which is proportional to strap 909 shortening length interval, is read by encoder 937.

Relaxation phase. The relaxation phase is effectuated by reactivating motor 950 for a second short time period whereby allowing further rotation of shaft 946 this time for bringing release disk 960 to a position where the disk teeth engage with gear 921 to unlock mainspring 916 from ratchet mechanism 924, thereby allowing further relaxation of mainspring 916 by counterclockwise rotation of disk 921. As the torque exerted on disk 926 by mainspring 916 decreases, the force exerted by the limb muscles which acts to increase the strap effective length combined with the opposite torque of strap return spring 936, cause disk 926 to rotate counterclockwise for relieving excessive strain in the system. Thus, unlocking clutch 920 immediately results not only with relaxation of mainspring 916 to its initial position but also with immediate fast lengthening of strap 809 to the relaxation effective length, through rotation of gears 926, 928, 930, 934 and 940 to resume their pre-loading positions as well as to rotate rollers 910 and 912 to pre-loading position. The relaxation of all components to pre-loading state also brings clutches 920 and 932 to their initial position, i.e., to be locked again and the cycle loading-shortening-relaxing starts all over again.

FIG. 9E illustrates an example of a ratchet mechanism 924 in a time sequential fashion for demonstrating the ratchet mechanism operation. Ratchet mechanism 924 comprises ratchet body 980 affixed to base plate 904 of case 901, a pawl 982 pivotally mounted on axis 984 within a recess of body 980 allowing a limited rotation of pawl 982 within the recess, and a spring 986 biased to pull pawl 982 toward the base plate. The free end of pawl 982 is engaged with inclined teeth 925a of ratchet gear 925. As can be clearly seen in sequence steps I-VI, ratchet mechanism 924 allows only for clockwise rotation of wheel 925 by pushing up the free end of pawl 982 (Steps I-IV) while counterclockwise rotation (steps V-VI) is hindered as teeth 925a press pawl 982 against body 980 preventing further rotation.

FIG. 9F illustrates an example of a clutch 932 for locking/unlocking gear 931 to body plate 904. The same clutch with minor modifications can serve also as clutch 920 for coupling/decoupling mainspring 916 and ratchet wheel 925. Steps I-VII are shown as cross sections through clutch 932 in the plane perpendicular to the rotation axis. Clutch 932 comprises an inner cylindrical part 992 having three half-circle recesses 992a at its outer perimeter, an outer ring 996 having three elongated recesses 996a at its inner perimeter, and a segmented annular element 994 interposed in the space there between. Elements 992, 994 and 996 are arranged concentrically around axis 915. Three circular rods 995 are interposed between adjacent segments of annular element 994. Rods 995, not connected to any of the other parts, can be pushed in the radial direction to occupy either recesses 992a or 996a but are always confined by segments 994. Outer ring 996 is connected to one end 998a of spring 998, having its second end 998b fixedly connected to case 901 biasing ring 998 counterclockwise. The outer perimeter of ring 996 is provided with tooth 996b to be engaged with double-spike 971 of cam 970. Elements 994 and 992 are each being an integral part of one of the two parts to be coupled or decoupled. By way of example, element 994 is perpendicularly extending from frontal body wall 904 while cylindrical element 992 is perpendicularly extending from the center of gear 931. Thus, when clutch 932 couples between elements 992 and 994, gear 931 is locked to the body 901. Step I of FIG. 9E shows clutch. 932 in the locked position. In this position, rods 995 are pressed by outer ring 996 into recesses 992a, preventing rotation of cylindrical part 992 in either direction. Double-spike 971 of cam 970 is directed away from clutch 932. In step II, double-spike 971 of cam 970 approach tooth 996b to engage the tooth 996b in steps III and IV and to rotate ring 996 clockwise. The rotation of ring 996 relative to fixed element 994 advances recesses 996a toward rods 995 such that cylindrical part 992 can rotate counterclockwise pushing rods 995 into recesses 996a, thus unlocking gear 931 to partly release the strain built up in the system during the loading phase. The rotation of gear 931 stops (step V) when further contraction of the strap is hindered by the limb resistance, preventing further rotation of gears 930 and consequently of gear 931 (see shortening phase description above). After double-spike 971 passes tooth 996b, ring 996 is again biased by spring 998 to rotate counterclockwise, as shown in step VI. However, rotation of ring 996 is prevented by rods 995 now partly positioned in recesses 996a. Thus, clutch 932 remains uncoupled allowing free rotation of cylindrical part 992. Referring to the relaxation phase description above, after clutch 920 is unlocked as well, all excessive strain in the system is released resulting in relaxation of the strap through counterclockwise rotation of gear 930 and consequently clockwise rotation of gear 931 and of element 992 as shown in step VII. The rotation of element 992 causes rods 995 to be pushed back into recesses 992a by outer ring 996 now free to rotate, as shown in step VIII, and clutch 932 returns to the locked position of step I.

It will be realized by persons skilled in the art that the specific construction of the ratchet and clutch mechanisms shown in FIGS. 9E and 9F are given by way of example only and that other equivalent mechanical elements having the same mechanical function can be used without departing from the scope of the invention.

As mentioned above, embodiment 900 is controlled by a microprocessor. The microprocessor controls motors 914 and 954 for timing the transitions between relaxed and contracted states in accordance with input parameters given by the user and the readings received from encoders 927, 958 and 937. A typical user interface is shown in FIG. 9F. User interface 500 includes a parameters keyboard 502, an alphanumeric keyboard 504 for entering desired values, a display panel 506 and an on/off switch 508. In parameters keyboard 502, Ta stands for the duration of relaxed phase; Tc for duration of contracted phase; F is the Force of mainspring 916; Tb is the transition time from relaxed to contracted state; Td is the transition time from contracted to relaxed state; and Xb is the change of the effective length of the strap between relaxed and trained states. Prior to operation, the user enters the values of Ta, Tc and F. The values of Tb, Td and Xb cannot be determined by the user and can be only measured by the encoders. During operation the actual values of these parameters as well as Tb, Td and Xb as measured by the encoders are displayed in display panel 906, each value next to corresponding parameter.

The embodiment illustrated through FIGS. 9 provides for enhanced flexibility by allowing choosing independently different parameters of the strap contracting-relaxing cycle. As such, embodiment 800 is particularly suitable as an experimental prototype device for deriving optimized parameters for different conditions and/or users. Embodiment 900 may also be used as a multi-user device by medical personnel for adjusting optimal parameters to each user. However, it will be realized that a lower cost mechanically-controlled version of embodiment 900, which is having the same main contraetion-relaxation mechanism as of embodiment 900, but is driven by only one continuously operating motor instead of two, may also be constructed.

It will be realized that both devices 800 and 900 can be designed to allow various cycle patterns adapted for the increasing of arterial flow from the heart to the limb or of venous flow from limb to heart. It will be also realized that one or more decelerating mechanisms can be coupled to the mechanism of devices 800 and 900 for controlling the transition time of at least one of the transitions. Such a slowing mechanism can be for example an impeller type mechanism. The de-accelerating mechanism allows for precise control of the pressure gradient profile during the transition. For example, the pressure can be controlled to reach the target value in a smooth monotonous way or to transiently overshoot the target value. Thus, a device in accordance with the invention may have fast pressure build up and slow pressure release, suitable for example for reducing the risk of DVT, or slow build up and fast release for enhancing arterial flow by inducing a venous suction effect. The effect, referred to as ‘suction effect’, is produced by the rapid fall in pressure at the end of each pressure cycle which causes the pressure at the veins to drop below normal and thus facilitates fast perfusion through distal tissues. This effect, referred to as ‘suction effect’, enables better distal tissue perfusion with or without high arterial pressure as is demonstrated below. Thus, in order to increase the flow to the peripheries, the device is tuned to build up pressure on the limb in order to compress the veins, and to rapidly release that pressure. Preferably the transition time from high to low pressure is of less than one 1 sec, more preferably of less than 300 mSec, 100 mSec, 30 mSec, or 10 mSec.

Typical operational parameters for inducing suction effect and enhancing arterial flow are: pressure at compressed state higher than 15 mmHg, preferably in the range of 15-180 mmHg, more preferably in the range of 30-120 and most preferably in the range of 60-100 mmHg; full cycle in the range of 0.5-300 sec, preferably in the range of 2-120 sec, more preferably in the range of 5-75 sec, most preferably in the range of 10-30 sec; duration of compressed phase less than 15 sec, preferably less than 8 sec, more preferably less than 1.5 sec or less than 300 msec; transition time from compressed to relaxed state less than 3 sec, preferably less than 1 sec, more preferably less than 200 msec and most preferably less than 100 or 30 msec; and transition time from relaxed to compressed state in the range of 100 msec-3 sec.

Typical operational parameters for enhancing venous flow for reducing the risk of DVT are: pressure at compressed state higher than 15 mmHg, preferably in the range of 15-120 mmHg, more preferably in the range of 25-60 and most preferably in the range of 30-50 mmHg; total cycle more than 5 sec, preferably in the range of 15-300 sec, more preferably in the range of 30-150 sec, most preferably in the range of 40-80; duration of compressed phase of less than 15 sec, preferably less than 8 sec, more preferably less than 3, most preferably less than 1.5 msec; transition time from relaxed to compressed state less than 10 sec, preferably less than 3 sec, more preferably less than 1 and most preferably less than 200, 100 or 30 mSec;

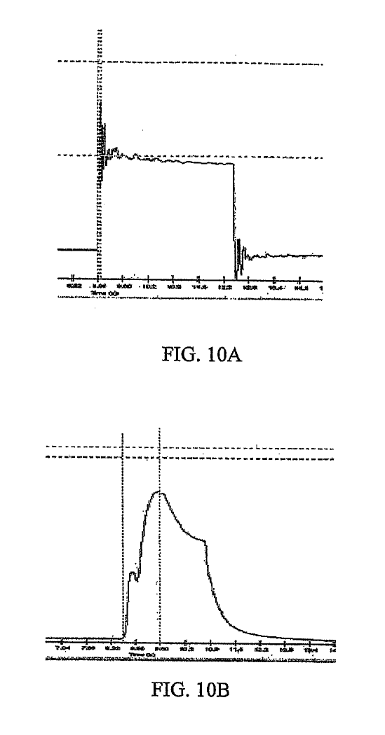

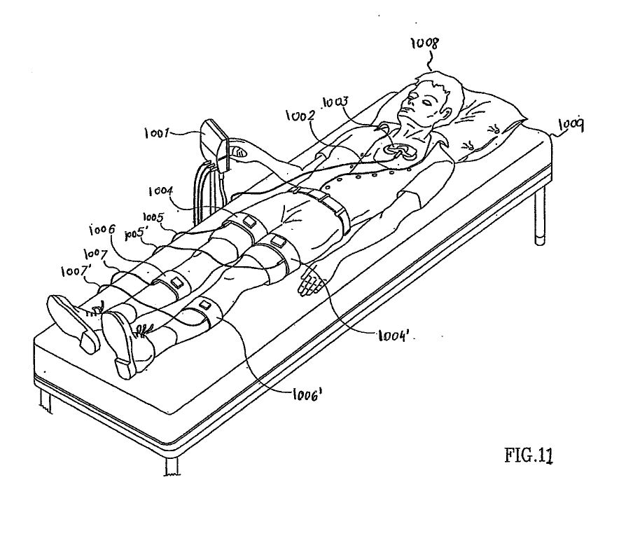

FIG. 10A is a typical pressure profile obtained by applying an instrument in accordance with embodiment 900 of the present invention showing the rise and fall of the pressure as function of time. For comparison sake, FIG. 10B shows a pressure profile, on the same time scale as of FIG. 10A, obtained by a typical commercially available IPC (intermittent pneumatic compression) instrument (Aircast VenaFlow). Both instruments were adjusted to converge to a similar pressure. As can be clearly seen, the pressure rise and fall times obtained by the present invention are much shorter than those obtained by the conventional pneumatic device. It can be also seen that the pressure profiles of the two instruments differ significantly. In accordance with the measurements shown in FIGS. 10A and 10B, it takes only about 0.06 seconds for the present apparatus to reach the maximum pressure value and about 0.08 seconds for the pressure to drop to its baseline value, while for the IPC device it takes about 0.96 seconds to reach the maximum pressure, about 0.68 seconds to drop to 75% of the maximum value and about 4.6 seconds to reach its baseline value. It will be realized that the pressure profile given in FIG. 10A is an example only and that the rise and fall times, as well as the transient gradient during pressure build up and pressure drop, can be easily varied by varying mechanical parameters of the device.

Referring now to FIG. 11, showing a person using a preferred embodiment of the external counter pulsation system. A person 1008 is lying comfortably on a standard domestic or hospital bed 1009. An ECG sensor 1003, such as Smart Q ECG sensor manufactured by Smart Q Technology or the Vernier ECG sensor produced and manufactured by Vernier Software & Technology, of 13979 SW Millikan Way Beaverton, Oreg. 97005-2886, U.S.A., comprising two electrodes (not shown) is attached to the chest of the patient, connected by a cable 1002 and transmitting information to a control unit 1001. Other sensors (not shown) can be attached to the person 1008, including a temperature sensor, a blood pressure sensor, a pulse sensor, and a sweat sensor for providing other physiological parameters to control unit 1001. Multiple compressing elements 1004, 1004′, 1006, 1006′ are attached to the patient's extremities at different locations, and are connect to control unit 1001 through cables 1005, 1005′, 1007, 1007′, respectively. Compressing elements 1004, 1004′, 1006, 1006′ generally comprise a limb attachment, said limb attachment comprises an actuator and a substantially flat encircling member that surrounds substantial part of the limb it is attached to. The encircling member can be a strap, a flap, a closure, a sleeve, or the like. Optionally, compressing elements 1004, 1004′, 1006, 1006′ comprise also one or more sensors, transmitting data such as, but not limited to, pressure or force applied to the limb of the patient, the temperature at the limb, and the like. Detailed embodiments of the compressing elements and the actuators, including the internal mechanisms and operating modes of operation are disclosed hereinabove and hereinafter, and in Israeli patent application Ser. No. 164286 titled A PORTABLE DEVICE FOR THE ENHANCEMENT OF CIRCULATION filed on 26 Sep. 2004, the full content of which is incorporated herein by reference. Control unit 1001 analyzes the data provided by ECG sensor 1003, and accordingly sends control commands to the actuators of elements 1004, 1004′, 1006, 1006′ which in turn instruct the actuators to pull and release the encircling members. The pulling and releasing of elements 1004, 1004′, 1006, 1006′ compress and release respectively the limbs of the patient. The compression and releasing of the limbs squeezes the limbs' muscles and increases peripheral blood and lymph flow in the limb. However, in order to modulate cardiac blood flow as well, pressing and releasing is performed in accordance with the patient's physiological condition, and more specifically with the heart beat cycle. The device of the present invention provides a pressing movement to the limb of the patient during the diastole, and release during the systole, thus increasing blood pressure. The device applies pressure such as to modulate cardiac blood flow by increasing the arterial and coronary pressure during the diastole. Alternatively, the device applies pressure such as to modulate cardiac blood flow by increasing the aorta and peripheral arteries pressure during the systole. The device can improve peripheral circulation such as to reduce peripheral resistance. The device operation applies pressure with parameters set to improve venous return. The device can also apply pressure gradients such as to promote vasodilatation. The operation of the device results in the application of falling pressure gradient such as to produce venous suction effect. The device applies pressure with high gradients such as to create sheer forces along the arteries, promoting coronary dilatation. Pressure can be applied in one or several locations along the limbs with the pressure applied substantially in a synchronized fashion. The compressing elements are preferably located in multiple locations along the limbs of the patient, for example around the calves, the lower thighs, and the upper thighs. The operation of the device results in the application of falling pressure gradient such as to produce venous suction effect. The device applies pressure with high gradients such as to create sheer forces along the arteries, promoting coronary dilatation. Pressure can be applied in one or several locations along the limbs with the pressure applied substantially in a synchronized fashion. The compressing elements are preferably located in multiple locations along the limbs of the patient, for example around the calves, the lower thighs, and the upper thighs. The operation of the device results in the application of falling pressure gradient such as to produce venous suction effect. The device applies pressure with high gradients such as to create sheer forces along the arteries, promoting coronary dilatation. Pressure can be applied in one or several locations along the limbs with the pressure applied substantially in a synchronized fashion. The compressing elements are preferably located in multiple locations along the limbs of the patient, for example around the calves, the lower thighs, and the upper thighs. The rapid and precisely timed squeezing of the muscles sends an amount of blood that is greater than the amount normally flowing through the veins of the patient and travels toward the heart. The sequential compression ensures that the amount of blood traveling from the most distant calf muscles are reinforced rather than trapped by the second and third compressions of the sequence. The result of the combined compressions is an increase in venous return, and an augmentation, or enhancement of diastolic pressure, which improves myocardial perfusion. Blood and lymph flow is further enhanced as a result of the pulling and releasing of the flat members. Persons skilled in the art will appreciate that the location of the sensors as shown in association with FIG. 1 above are merely exemplary and the sensors can be located in various locations, including remotely from the body of the patient. Likewise, the control unit 1 can be connected to the various sensors discussed above via wireless means, such as infrared and blue tooth or RF sensors and emitters. The control unit 1 can be a small sized unit attached to the belt of the patient or even attached to the pulling and releasing elements.

Referring now to FIG. 12, showing a flowchart of the main steps of the disclosed method. At step 1030, the process starts, by the patient connecting himself, or being connected by another person to the device. Step 1030 comprises connecting the limb attachments to the user's limbs. The number and locations of the limb attachments are determined by a physician's recommendation or by the instructions provided with the device of the present invention. In general the device can be applied by the person through the opening of the flat member, attaching the device to the limb and closing the flat member so as to form a closure around the limb. In other embodiments, the flat members comprise of flaps and the person using the device will attach the device to the limb fixing the flaps onto the skin of the limb enabling the squeezing of said limb. Next, the ECG sensors are attached to the patient's chest. In an alternative embodiment, the ECG or other sensors, such as temperature sensor, blood pressure sensor, pulse sensor, optic sensor, sweat sensor or any other sensor that is able to provide physiological parameters is attached to the person's chest or other relevant body parts from which a measurement can be taken and provided to the control unit. At step 1034, the system optionally performs a self test, in which its proper functionality and proper connections are tested. If an error is detected during the self test, or at anytime later in further tests or during the operation, the system issues an appropriate message and possibly suggestion for correction, such as “Reconnect ECG cable”. At step 1038, the compressing elements are tested for proper functioning and connection. Then, at step 1042, the ECG device and its connections and in addition any other sensor connected to the device are tested. Additionally, the physical condition of the patient is evaluated. If relevant parameters such as his temperature, blood pressure, heart rate blood vessels; movement; sweat amount; sweat composition or any other physiological parameter which may suggest that the device of the present invention should not be used or that the device should be used with varying parameters taking into account the condition of the person, is out of the normal range, a proper message or alert is generated. Once the functionality of the system and the condition of the patient have been established, a treatment plan is optionally presented to the patient at step 1046, and once the patient approves at step 1048, the treatment starts. According to the data transmitted by the ECG sensor, and the other sensors, the device determines the heart beat cycle at step 1052, including the systole and diastole periods. Optionally, the device further monitors the temperature, pulse and blood pressure of the patient throughout the use of the device. Then at the diastole, step 1056 is performed in which the device transmits constriction commands to the limb attachment for a predetermined period of time, such as between a few milliseconds and up to tens of seconds, and then transmits a command to release. The release can be a quick release performed over a few milliseconds or a slow release performed over a number of seconds to tens of seconds. The constriction and release can also be measured by heart beat cycles and can be at a length of about 1 to about 200 heart beats. The constriction command is translated into a pulling of the flat member of the limb attachment by the actuator of the device. The releasing command is translated into the release of the flat member of the limb attachment. This process can be repeated for a predetermined period of time, or for a predetermined number of heart beat cycles in step 1060. In accordance with a predetermined plan, or as a result of a user or patient's request or the request of a third party such as a care giver or a doctor or a supervisor, the system stops and the patient is disconnected form the system at step 1064. In accordance with another embodiment of the present invention, the constriction and release of the limb attachment flat member can be associated with other physiological parameters of the patient, such as the temperature, blood pressure and pulse.

Referring now back to FIGS. 2A and 2B, showing an exemplary compressing element. The exemplary element comprises an actuator housing or frame 25, a belt or strap 1, and a buckle 68. One end of belt 1 resides inside frame 25, and a second end 65 folds around a vertical opening in buckle 68. The other end of buckle 68 is permanently inserted into housing 25. End 65 of belt 1, is attached back to belt 1, by any attachment mechanism, such as a pair of Velcro patches, adhesive area, a buckle and the like. Internal area 50 is substantially surrounded by belt 1, buckle 68, and housing 25. In other embodiments, the frame is connected to a pair of flaps which compress the limb of the patient. The flaps do not form a full closure over the limb, rather provide an opening which enables a quick application and removal of the device to and from the limb of the patient. Housing 25 further comprises an on/off switch 6 for activating and deactivating the element. Switch 6 can be located on the control unit 1001 of FIG. 11 or can be located in a separately designed device connected by wire or wirelessly to the compressing elements of FIG. 11. When the element is used in the context of the current invention, it further comprises a cable connecting to a control unit and a plug (not shown) connecting the cable to housing 25. As noted above the compressing elements of FIG. 11 can be wirelessly connected to the control unit 1001 of FIG. 11. In the context of the current invention, on/off switch 6 is turned on to enable the unit to be controlled by the control unit. When switch 6 is turned off, activation of the unit is disabled. When the unit receives an activation signal through the cable or wirelessly, the internal mechanism residing in housing 25 (not shown) pulls belt 1, thus shortening the available length of belt 1, reducing internal area 50, and activating pressure on the limb surrounded by the device. When the unit receives a deactivation signal, the belt is released and the pressing of the limb is reduced or stopped. The internal mechanism inside housing 25 comprises a motor and mechanical elements such as rods, tooth wheels, eccentric wheels, and the like. Preferred embodiments for the internal mechanism inside housing 25 and other preferred embodiments are detailed hereinabove and in Israeli patent application Ser. No. 164286 titled A PORTABLE DEVICE FOR THE ENHANCEMENT OF CIRCULATION filed on 26 Sep. 2004, the full content of which is incorporated herein by reference.

The disclosed invention provides an easy to use, cheap, non irritating device and method for modulating cardiac blood flow, by intermittently compressing the limbs of the patient in accordance with his or her heart beat cycle. The device is quite, efficient, does not require special space or equipment and can be used at the patient's home, and not necessarily in a health institute. According to another preferred embodiment of the present invention a device such as the devices described above is attached to a limb, for modulating cardiac blood flow by intermittently applying rapid pressure on the limb. The limb attachment substantially surrounds at least most of the limb perimeter in at least one location. The device can further include at least one mechanical actuator. The intermittently rapid pressure is generated by shortening one or more of the flat members surrounding the limb. Alternatively, the intermittently rapid pressure is generated by pressing with one or more movable plates, or by pressing with one or more flap moving in and out of a housing.

If an intermittent pressure is applied, the intermittent pressure application and the pressure release are preferably synchronized. The delay between intermittent pressure applications can be less than 500 millisecond, or less than 100 millisecond, or the like. The intermittent pressure can be applied sequentially. In such case the sequence preferably starts from distal regions and continues to proximal regions. The commands issued by the control unit of the device as is noted herein and above can be responsive to physiological events. The device can further comprise an analysis unit that analyzes the physiological input and controls the device activity accordingly. The parameters to be tuned according to this embodiment can comprise of any of the following: pressure levels, pressure durations, no-pressure duration, pressure rise time, pressure fall time, pressure delays among locations, pressure delay from physiological event, rest periods, sequence of pressure levels, and the like.

The device can be connected to at least one external sensor. The device can include one or more sensors. The sensors are located within the device adjacent to the body of the patient or are mounted in other locations on the person's body or in the persons' vicinity in order to allow clear sensor input. The physiological input is sensed by at least one device such as electrocardiograph, photoplathismography, or any other relevant sensor device. The physiological input is one or more of the following: heart rate; blood pressure; blood vessels; movement; pulse; sweat amount, sweat composition, temperature, and the like. The input through the sensors suggest a physiological event, which can be used as a trigger for device pressure application. Thus, according to a physiological event a predetermined pressure is applied to the limb. For example, the pressure can be applied in synchrony with the heart beat. In accordance with another example, the device applies pressure according to timing mechanism not in synchrony with heart beat. In accordance with yet another example, the device applies pressure every predetermined number of heart beats.

The device can be located on any of the following limbs or areas of the body: foot, calf, hip, arm and the like. The device can be used for treating heart failure, coronary disease, or angina pectoris. The operation of the device is preferably such that the device presses the limb by applying a pre-determined force or a pre-determined pressure. Actuators located on the device are attached to the patient. The device volume necessary for of actuator and all machinery used to produce the pressure at each of the location is preferably less than 2000 cc, or less than 1000 cc, or the like.

In one preferred embodiment the weight of all machinery used to produce the pressure at each of the at least one location is less than 3 Kg or less than 1.5 Kg or less than 1 Kg or lighter.

The pressing rise time of the device is preferably substantially less than 1 second or less than 300 milliseconds or less than 100 milliseconds or less than 50 milliseconds or the like. Alternatively, the pressing fall time substantially less than 1 second or less than 300 milliseconds or less than 100 milliseconds or less than 50 milliseconds or the like.

In accordance with a preferred embodiment of the device, the pressing produces pressure within the range of 15-400 mmHg, or 25-150 mmHg or 40-100 mmHg, or the like. The pressing duration can be less than 3 seconds with varying times of for example 1 sec, 300 milliseconds, 100 milliseconds, and the like.

In accordance with another embodiment of the present invention, the device can include at least one data logging unit. The data logging unit can store activity information comprising time, pressure, usage information. The device can store physiological data. The device can include an analysis unit enabling to review the data and analyze it.

Persons skilled in the art will appreciate the fact that many alternatives exist to various parts of the device. The number and locations of the compressing elements can vary according to the patient's condition and other factors as determined by a physician. The elements can be placed symmetrically on the patient's right and left limbs or non symmetrically. The pressure can be applied to all the elements simultaneously, or at different times, such as for example, starting at the distally located elements and continuing to the more proximally located elements. The elements can activate the same or different pressures on the limbs, with same or different pressure rise and fall rates. Other characteristics of the employed protocols can be the same or different among the elements as well, such as activation times, duration, and the like. Additionally, each compressing member can include multiple pressing elements, such as multiple parallel belts, each belt activating pressure simultaneously or non-simultaneously with the other belts.

Various types of compressing elements can be employed, including but not limited to the types discussed above. The compressing elements can further comprise a sleeve-like member for wrapping larger areas of the limbs. The sleeves can have an inner volume and contain fluid or gel for even distribution of the pressure over a larger area. Alternatively, the compressing elements can be disposable. Each compressing element can further comprise a pressure, force, or temperature sensor, for monitoring the effect of the compression on the patient. Other sensors can be used instead of, or in addition to an ECG device. Any off-the-shelf or tailor-made product, that can distinct diastole and systole can be used. Alternatively, other parameters related to the physiological condition of the patient can be used as well, such as blood pressure, temperature perspiration composition and the like. The components of the system, namely the sensor, the control unit and the compressing elements can communicate via different communication equipment and protocols, including wireless communication. The control unit can further include transmission components for logging or transmitting the data received from the ECG and from the compression elements, and the activities taken by the system to another, possibly remote location, for purposes such as control, logging, and the like. Alternatively, the system can comprise a separate logging device to log the physiological data, the data of provided by the sensors of the elements or the pressing and releasing activities. The control unit can her comprise a receiver device, for receiving instructions form a remote location, such as a remotely present physician. In yet another embodiment, the control unit can store treatment plan for multiple patients, and activate the correct treatment plan once the patient identified himself. Other embodiments can comprise the possibility for a remote positioned physician or other personnel to remotely control the operation of the system (e.g. on/off command), monitor (e.g. telemetry) efficiency and safety of the system. According to other embodiments the remotely positioned control unit can comprise a billing unit for billing the use of the system or services of a physician.

It will be appreciated by persons skilled in the art that the present invention is not limited to what has been particularly shown and described hereinabove. Rather the scope of the present invention is defined only by the claims which follow.

What is claimed is:

1. A system for modulating cardiac blood flow of a patient by intermittently applying and releasing pressure on a limb of the patient, the device comprising:
at least one limb attachment, the limb attachment comprises an at least one generally encompassing member substantially surrounding the limb perimeter in at least one location, at least one actuator for pulling and releasing the flat member;
at least one processing unit for issuing commands to the at least one actuator.

2. The system of claim 1 wherein the commands are determined based on an at least one physiological parameter of the patient.

3. The system of claim 1 further comprising an at least one connection to an external sensor measuring the at least one physiological parameter of the patient.

4. The system of claim 1 further comprising an at least one sensor for measuring at least one physiological parameter of the patient.

5. The system of claim 1 where the actuator is a mechanical actuator.

6. The system of claim 1 wherein the limb attachments are attached to any pf the following: a foot, a calf, a shin, a hip, an upper arm, a forearm.

7. The system of claim 1 wherein the system comprises at least one pair of limb attachments.

8. The system of claim 7 where each pair of limb attachments is symmetrically attached to the body of the patient.

9. The system of claim 1 wherein the actuators are attached to the patient.

10. The system of claim 1 where the at least one encircling member is a strap, a flap, a closure, or a sleeve.

11. The system of claim 2 wherein the physiological parameter is sensed by an electrocardiograph or photoplathismography device.

12. The system of claim 2 wherein the physiological parameter is at least one of the following: heart rate; blood pressure; blood vessels; movement; temperature; sweat amount; sweat composition.

13. The system of claim 1 wherein the device applies pressure such as to modulate cardiac blood flow by increasing the arterial and coronary pressure during the diastole.

14. The system of claim 1 wherein the device applies pressure such as to modulate cardiac blood flow by increasing the aorta and peripheral arteries pressure during the systole.

15. The system of claim 1 whereby the device improves peripheral circulation such as to reduce peripheral resistance.

16. The system of claim 1 wherein pressure is applied to improve venous return.

17. The system of claim 1 wherein the device applies pressure gradients such as to promote vasodilatation.

18. The system of claim 1 wherein the device applies falling pressure gradient such as to produce venous suction effect.

19. The system of claim 1 wherein the system applies pressure with high gradients such as to create sheer forces along the arteries, promoting coronary dilatation.

20. The system of claim 1 wherein the system applies pressure in several locations along the limbs wherein the pressure is applied substantially in a synchronized fashion.

21. The system of claim 1 wherein the intermittent pressure applications are synchronized.

22. The system of claim 1 wherein the intermittent pressure releases are synchronized.

23. The system of claim 1 wherein the delay between intermittent pressure applications is less than 500 milliseconds.

24. The system of claim 1 wherein the delay between intermittent pressure applications is less than 100 milliseconds.

25. The system of claim 1 wherein the intermittent pressure is applied sequentially.

26. The system of claim 25 wherein the pressure application starts from distal regions.

27. The system of claim 2 wherein the system is responsive to an at least one physiological event.

28. The system of claim 27 wherein the at least one physiological event is used as a trigger for applying and releasing intermittent pressure on the limb of the patient

29. The system of claim 27 wherein the processing unit analyzes the at least one physiological parameter and controls the system activity accordingly.

30. The system of claim 1 wherein the commands issued by the processing unit relate to any of the group made of: pressure levels, pressure durations, no-pressure duration, pressure rise time, pressure fall time, pressure delays among locations, rest periods, sequence of pressure levels.

31. The system of claim 2 wherein the commands issued by the processing unit relate to a pressure delay from physiological event.

32. The system of claim 1 wherein the at least one limb attachment further comprises a sensor.

33. The system of claim 32 wherein the sensor is an external sensor.

34. The system of claim 32 where the sensor senses pressure or force or temperature or impedance or plethysmography.

35. The system of claim 34 wherein the pressure or the force or the temperature are used in determining the commands issued by the processing unit.

36. The system of claim 1 wherein the commands are issued according to a timing mechanism.

37. The system of claim 2 wherein the pressure is applied according to the heart beat of the patient.

38. The system of claim 2 wherein the pressure is applied every predetermined number of heart beat cycles.

39. The system of claim 1 wherein the system is used for treating heart failure.

40. The system of claim 1 wherein the system is used for treating coronary disease.

41. The system of claim 1 wherein the system is used for treating angina pectoris.

42. The system of claim 1 wherein the system applies pressure on the limb by applying a pre-determined force.

43. The system of claim 1 wherein the system applies pressure on the limb by applying a pre-determined pressure.

44. The system of claim 1 wherein the volume required for the actuator used to produce the pressure at each of the at least one limb attachments is less than 2000 cc.

45. The system of claim 1 wherein the volume required for the actuator used to produce the pressure at each of the at least one limb attachments is less than 1000 cc.

46. The system of claim 1 wherein the weight required for the actuator used to produce the pressure at each of the at least one limb attachments is less than 3 Kg.

47. The system of claim 1 wherein the weight required for the actuator used to produce the pressure at each of the at least one limb attachments is less than 1.5 Kg.

48. The system of claim 1 wherein the weight required for the actuator used to produce the pressure at each of the at least one limb attachments is less than 1 Kg.

49. The system of claim 1 wherein the pressure rise time is less than 1 second.

50. The system of claim 1 wherein the pressure rise time is less than 300 milliseconds.

51. The system of claim 1 wherein the pressure rise time is less than 100 milliseconds.

52. The system of claim 1 wherein the pressure rise time is less than 50 milliseconds.

53. The system of claim 1 wherein the pressure fall time is less than 1 second.

54. The system of claim 1 wherein the pressure fall time is less than 300 milliseconds.

55. The system of claim 1 wherein the pressure fall time is less than 100 milliseconds.

56. The system of claim 1 wherein the pressure fall time is less than 50 milliseconds.

57. The system of claim 1 wherein the pressure is within the range of 15-400 mmHg.

58. The system of claim 1 wherein the pressure is within the range of 25-150 mmHg.

59. The system of claim 1 wherein the pressure is within the range of 40-100 mmHg.

60. The system of claim 1 wherein the pressure application duration is less than 3 seconds.

61. The system of claim 1 wherein the pressure application duration is less than 1 second.

62. The system of claim 1 wherein the pressure application duration is less than 300 milliseconds.

63. The system of claim 1 wherein the pressure application duration is less than 100 milliseconds.

64. The system of claim 1 wherein the actuator comprises at least one motor.

65. The system of claim 1 wherein the actuator comprises an at least one mechanical element.

66. The system of claim 65 where the at least one mechanical element is any of the following group: a rod; a tooth wheel; an eccentric wheel.

67. The system of claim 1 wherein the actuator comprises at least one eccentric wheel.

68. The system of claim 1 wherein the system includes at least one data logging unit.

69. The system of claim 68 wherein the data logging unit stores activity information of the system comprising time, pressure, usage information.

70. The system of claim 2 wherein the system includes at least one data logging unit.

71. The system of claim 70 wherein the data logging unit stores the at least one physiological parameter.

72. The system of claim 1 further comprising an analysis unit for data reviewing and analysis.

73. The system of claim 3 further comprising an analysis unit for data reviewing and analysis.

74. The system of claim 4 further comprising an analysis unit for data reviewing and analysis.

75. The system of claim 4 further comprising an unit for data telemetry.

76. The system of claim 4 further comprising an unit for on and off command of the system.

77. A method for modulating cardiac blood flow of a patient by applying intermittent pressure on a limb of the patient, the method comprising the steps of:
issuing an at least one command to an at least one actuator associated with an at least one limb attachment, said at least one actuator causing the beginning or end of application of pressure by the at least one limb attachment on the limb, thereby changing the circumference of the limb of the patient thus modulating the cardiac blood flow of the patient;
executing said command by the at least one actuator.

78. The method of claim 77 wherein the method further comprises the following steps:
receiving an at least one physiological parameter from an at least one sensor associated with the patient;
determining the beginning of diastole based on the at least one physiological parameter received from the at least one sensor associated with the patient

79. The method of claim 77 wherein the pressure is applied only during systole.

80. The method of claim 77 wherein the pressure is applied symmetrically to the body of the patient.

81. The method of claim 77 wherein the pressure is applied to the body of the patient starting at distal limb attachments.

82. The method of claim 77 wherein the pressure is applied by shortening of one or more straps circulating the limb.

83. The method of claim 77 wherein the pressure is applied by pressing with one or more movable plates.

84. The method of claim 77 wherein the pressure is applied by pressing with one or more flap moving in and out of a housing.

85. The method of claim 77 further comprising a step of storing data.

86. The method of claim 85 where the data includes activity information comprising time, pressure and usage information.

87. The method of claim 85 wherein the data includes physiological data.

88. The method of claim 77 further comprising the step of measuring the at least one physiological parameter.

89. The method of claim 77 wherein the application of pressure by the at least one limb attachment on the limb is performed intermittently.

90. The method of claim 77 further comprising a step of evaluating the physical condition of the patient prior to issuing the at least one command.

91. The method of claim 77 wherein the method improves the blood flow of the coronary artery.
